(19) United States
(12) Patent Application Publication
(10) Pub. No.: US 2025/0288612 A1
(43) Pub. Date: Sep. 18, 2025

(54) CHIMERIC ANTIGEN RECEPTOR AGAINST PROGRAMMED DEATH LIGAND 1 (PD-L1) AND APPLICATION THEREOF

(71) Applicants: Topmunnity Therapeutics Taiwan Limited, Taipei City (TW); ARCE therapeutics, Inc., Taipei City (TW)

(72) Inventors: Jyun WANG, Taipei City (TW); Chao-Yua HUANG, Taipei City (TW); Chien-Tsun KUAN, Cary, NC (US); Kao-Jean HUANG, Hsinchu County (TW); Kuan-Yu LAI, New Taipei City (TW)

(73) Assignees: Topmunnity Therapeutics Taiwan Limited, Taipei City (TW); ARCE therapeutics, Inc., Taipei City (TW)

(21) Appl. No.: 19/079,956

(22) Filed: Mar. 14, 2025

Related U.S. Application Data
(60) Provisional application No. 63/565,116, filed on Mar. 14, 2024.

(51) Int. Cl. A61K 35/17; A61K 40/11; A61K 40/15; A61K 40/31; A61K 40/42; A61P 35/00; C07K 14/705; C07K 14/725; C07K 16/28; C12N 5/0783

(52) U.S. Cl. CPC A61K 35/17; A61K 40/11; A61K 40/15; A61K 40/31; A61K 40/421; A61P 35/00; C07K 14/7051; C07K 14/70517; C07K 14/70578; C07K 16/2827; C12N 5/0636; C12N 5/0646; A61K 2239/13; A61K 2239/17; A61K 2239/21; A61K 2239/49; C07K 2317/24; C07K 2317/53; C07K 2317/565; C07K 2317/622; C07K 2319/02; C07K 2319/03

(57) ABSTRACT

The present invention relates to a chimeric antigen receptor (CAR) against programmed death ligand 1 (PD-L1) and application thereof. In particular, the CAR comprises an antigen-binding domain which binds to PD-L1; and the anti-PD-L1 CAR cells thus produced are useful in CAR cell therapy.

### RELATED APPLICATION

This application claims priority to U.S. Provisional Application No. 63/565,116, filed on Mar. 14, 2024, the content of which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 12, 2025, is named “20250312-TMT0002WO-Seq list.xml” and is 33,947 bytes in size. The sequence listing contained in this .XML file is part of the specification and is hereby incorporated by reference herein in its entirety.

### TECHNOLOGY FIELD

The present invention relates to a chimeric antigen receptor (CAR) against programmed death ligand 1 (PD-L1) and application thereof. In particular, the CAR comprises an antigen-binding domain which binds to PD-L1; and the anti-PD-L1 CAR cells thus produced are useful in CAR cell therapy.

### BACKGROUND OF THE INVENTION

Programmed Death-Ligand 1 (PD-L1) plays a pivotal role in modulating immune responses, both in normal physiological conditions and in cancer. In normal cells, PD-L1 contributes to immune homeostasis by preventing T cell overactivation and protecting against autoimmunity. However, in the context of cancer, PD-L1's overexpression on tumor cells inhibit the PD-1 pathway on T cells, leading to an impaired immune response and facilitating tumor immune evasion. This functionality of PD-L1 underpins its significance as a target in cancer immunotherapy^{1,2}.

The interaction between PD-L1 and PD-1 is intricately influenced by the glycosylation status of PD-L1. Specifically, the deglycosylation at the N35 site on PD-L1 significantly alters its binding affinity with PD-1, impacting the immunosuppressive activity of PD-L1 in cancer^{3,4}. Beyond the N35 site, PD-L1 possesses additional glycosylation sites at N192, N200, and N219, each playing a role in PD-L1's stability and function5. These glycosylation sites not only enhance PD-L1's structural integrity but are also crucial for its prolonged interaction with immune cells, contributing to its immune checkpoint functionality.

Studies have revealed that normal tissues and naive immune cells express very low levels of PD-L1 and glycosylated PD-L1 (gPD-L1), highlighting the potential for developing immunotherapies that minimize collateral damage to normal tissues³. Targeting gPD-L1 with specificity could significantly increase the safety profile of these therapies, sparing normal cells that express these molecules at low levels.

Interestingly, FDA-approved anti-PD-L1 antibodies such as Atezolizumab (Tecentriq), Durvalumab (Imfinzi), and Avelumab (Bavencio) demonstrate varying affinities for glycosylated versus deglycosylated forms of PD-L1. This disparity in binding affinity, particularly a reduced affinity for the deglycosylated form of PD-L1, is a critical consideration in the development and clinical application of these antibodies, influencing their effectiveness and specificity in targeting cancer cells⁴.

Chimeric Antigen Receptor T (CAR-T) therapy represents a groundbreaking advancement in cancer treatment, involving the reengineering of a patient's T cells to target and eradicate cancer cells. This innovative approach, however, faces significant challenges, notably toxicity concerns such as Cytokine Release Syndrome (CRS) and Immune Effector Cell-Associated Neurotoxicity Syndrome (ICANS), stemming from the extensive activation of CAR-T cells within the patient⁶.

Recent research⁷ has revealed that PD-L1-CAR-bearing T cells, developed using segments of the Atezolizumab sequence, can target and eliminate bead-stimulated T cells expressing PD-L1. This observation during the CAR-T production process's expansion phase underscores the potential for these CAR-T cells to also impact normal cells expressing PD-L1. This finding highlights the critical balance needed in CAR-T therapy development to target cancer cells effectively while minimizing unintended effects on normal tissue.

The expansion of CAR-T therapy to target antigens beyond CD19 introduces new challenges, including the risk of unforeseen toxicities. This necessitates thorough safety evaluations and meticulous development strategies to mitigate such risks⁸. In a notable instance, a clinical trial involving CAR-T therapy based on Atezolizumab, an anti-PD-L1 antibody, was conducted but terminated early after enrolling only one patient due to significant safety concerns. This trial's premature conclusion underscores the critical need for enhanced specificity and safety in CAR-T cell design, particularly when targeting antigens like PD-L1 that are also present on normal cells^{7,9}.

### SUMMARY OF THE INVENTION

The present invention provides a chimeric antigen receptor (CAR) against PD-L1, cells expressing the CAR and CAR cell therapy using the same. In particular, the CAR cells as produced exhibit a heightened specific cytotoxicity against PD-L1 expressing cells, and reduced cytotoxicity against non-target cells. Further, the CAR cells of the present invention exhibit effective fratricide resistance without undermining their on-target cytotoxicity.

Particularly, in one aspect, the present invention provides a CAR comprising a PD-L1 binding domain which comprises (a) a heavy chain variable region (V_{H}) which comprises a heavy chain complementary determining region (HC CDR1) of SEQ ID NO: 1, a heavy chain complementary determining region 2 (HC CDR2) of SEQ ID NO: 2, and a heavy chain complementary determining region 3 (HC CDR3) of SEQ ID NO: 3; and (b) a light chain variable region (V_{L}) which comprises a light chain complementary determining region 1 (LC CDR1) of SEQ ID NO: 4, a light chain complementary determining region (LC CDR2) of SEQ ID NO: 11, and a light chain complementary determining region 3 (LC CDR3) of SEQ ID NO: 6.

In some embodiments, the CAR further comprises a hinge domain, a transmembrane domain and an intracellular signaling domain.

In some embodiments, the CAR comprises the PD-L1 binding domain comprising, from N-terminus to C-terminus, the V_{H} and the V_{L} or the V_{L} and the V_{H}.

In some embodiments, the V_{H} and the V_{L} are linked by a linker.

In some embodiments, the intracellular signaling domain comprises at least one be selected from CD137 (4-1BB) signal domain, CD28 signal domain, CD27 signal domain, ICOS signal domain, CD3ζ signal domain, 2B4 signal domain and any combination thereof.

In some embodiments, the CAR comprises the amino acid sequence of SEQ ID NO: 19 or 20. In some embodiments, the CAR comprises the amino acid sequence of SEQ ID NO: 26 or 25.

The present invention also provides a nucleic acid molecule comprising a nucleotide sequence encoding a CAR as described herein. In some examples, the nucleic acid molecule is a vector.

The present invention also provides a cell comprising a nucleic acid molecule comprising a nucleotide sequence encoding a CAR as described herein. The cells are engineered to express the CAR on their surface which are useful in CAR cell therapy. In some embodiments, the cells are T cells. In some embodiments, the cells are NK cells. A pharmaceutical composition comprising a cell for expressing a CAR as described herein is also provided.

Further provided is an isolated antibody against PD-L1 (anti-PD-L1 antibody). Such antibodies exhibit specific binding affinity to the target PD-L1 antigen and are useful in preparing a CAR construct for cell therapy. In some embodiments, the PD-L1 antibody of the present invention has the amino acid sequences of the heavy chain and light chain CDRs as exemplified in Table 3.

In another aspect, the present invention also provides a method for treating a subject having a disease, disorder or condition associated with an elevated expression of a tumor antigen by administering to the subject an effective amount of a cell genetically modified to express a CAR or an antibody as described herein. The present invention also provides use of a cell genetically modified to express a CAR or an antibody as described herein for manufacturing a medicament for treating a subject having a disease, disorder or condition associated with an elevated expression of a tumor antigen. The present invention also provides a cell genetically modified to express a CAR or an antibody as described herein for use in treating a disease a subject having a disease, disorder or condition associated with an elevated expression of a tumor antigen.

In some embodiments, the tumor antigen is PD-L1.

In some embodiments, the subject is suffered from cancer.

In some embodiments, the cancer is selected from the group consisting of breast cancer, colon cancer, head and neck cancer, thyroid cancer, lung cancer, ovarian cancer, pancreatic cancer, gastric cancer, kidney cancer, glioblastoma and leukemia.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows the structure of CAR in the viral vector backbone plasmid. pTT003 comprises a human CD8 signal peptide, V_{L}, GS linker, V_{H}, Ala-Ala-Ala, human CD8a transmembrane domain, 4-1BB, and human CD3ζ. Herein, Alanine-Alanine-Alanine as a NotI restriction enzyme cutting site.

FIG. 2 shows a comparison of immunohistochemical staining between the monoclonal antibody mTT-01 and chimeric Atezolizumab in human placental tissue. The left image shows the staining result of mTT-01; the right image shows the staining result of chimeric Atezolizumab. The placenta, being fetal tissue, thus expressed higher levels of PD-L1 to prevent attack by maternal T cells. The arrows indicate that both antibodies clearly stained the cell membranes.

FIG. 3 shows a comparative immunohistochemical staining of the monoclonal antibody mTT-01 and chimeric Atezolizumab in human tonsillar tissue. The left image presents the staining result of mTT-01; the right image, that of chimeric Atezolizumab. The tonsil, a human organ tissue, exhibited higher PD-L1 expression. Both antibodies show a similar staining distribution, but the staining by mTT-01 was noticeably weaker than chimeric Atezolizumab.

FIG. 4 shows the comparative immunohistochemical staining of the monoclonal antibody mTT-01 and chimeric Atezolizumab in human breast cancer tumors. The left image shows the staining result of mTT-01; the right image, that of chimeric Atezolizumab staining result. Both antibodies displayed similar staining distribution, with chimeric Atezolizumab showing deeper staining in the cytoplasm, and mTT-01 showing deeper staining on the tumor cell membranes. Additionally, chimeric Atezolizumab showed staining in the stroma and nucleus of the breast tissue.

FIG. 5 shows a comparison of immunohistochemical staining between the monoclonal antibody mTT-01 and chimeric Atezolizumab in human tumors and adjacent normal tissues. Given the importance of CAR technology in enabling immune cells to recognize biomarkers on the cell surface, out of 43 tumor samples with cell membrane staining as positive, mTT-01 recognized 34 samples, yielding a positive recognition rate of 79%; chimeric Atezolizumab recognized 20 samples, with a positive recognition rate of 46.5%. Among 44 samples of tumor-adjacent normal tissue, mTT-01 showed cell membrane staining in only 3 samples, adjacent normal tissue recognition rate of 6.8%; chimeric Atezolizumab showed staining in 10 samples, adjacent normal tissue recognition rate of 22.7%. This indicated that mTT-01 had better specificity than chimeric Atezolizumab in cell membrane staining, potentially leading to lower toxicity to normal tissues.

FIG. 6 shows the establishment of CAR constructs in CD3+ T cells, specifically TT0003 CAR-T. Following the transduction of primary CD3+ T cells with a lentiviral vector, the surface expression of TT0003 CAR was assessed using PD-L1 staining by FACS. The left image serves as a reference for normal CD3+ T cells, outlining a cell distribution exceeding the threshold at 0.15%, thereby establishing the baseline CAR expression. In the middle image, the mock group (without transduction) demonstrates a staining of 0.35%. The right image illustrates the embodiment, where populations were divided with the same threshold, revealing over 99.7% of T cells expressing CAR, with an MFI of 35228. This value was significantly higher than the 31.8 MFI observed in normal CD3+ T cells, indicating successful TT0003 expression in CAR-T cells.

FIG. 7 shows MDA-MB-231 PD-L1^{KO} cell line which lacks PD-L1 expression. The MDA-MB-231 cell line expresses PD-L1 at a high level; the MDA-MB-231 PD-L1^{KO} cell line does not express PD-L1.

FIG. 8 shows the varying cytotoxicity of TT0003 CAR-T cells against MDA-MB-231-derived cell lines with different levels of PD-L1 expression. In the top panel, TT0003 CAR-T cells exhibited differential cytotoxic effects on the MDA-MB-231 cell line, depending on the E:T ratios and duration of co-culture. Conversely, the middle panel shows that TT0003 CAR-T cells did not elicit significant cytotoxicity against the MDA-MB-231 PD-L1^{KO} cell line. As a negative control, the bottom panel shows CD3+ T cells, which showed no substantial cytotoxicity against the parental MDA-MB-231 cell line.

FIG. 9 shows the cytotoxicity of TT0003 CAR-T cells on MDA-MB-231 GFP/Luc cells using a luciferase assay. In the top panel, both E:T ratios of 2:1 and 5:1 demonstrated efficacy in killing MDA-MB-231^{GFP/Luc} cells. Specifically, at an E:T ratio of 2:1 and after 24 hours of co-culturing, approximately 50% of MDA-MB-231^{GFP/Luc} cells were eliminated, while at both E:T ratios and after 24 or 48 hours of co-culturing, no viable MDA-MB-231^{GFP/Luc} cells remained. As a negative control, the bottom panel showed that CD3+ T cells not only exhibited no cytotoxicity against MDA-MB-231^{GFP/Luc} cells but also promoted a slight increase in the growth of MDA-MB-231^{GFP/Luc} cells after 48 hours of co-culturing.

FIG. 10 shows the PD-L1 expression levels on the cell surfaces of eight cancer cell lines, as detected by FACS. The cell lines include U87-MG (glioblastoma), SK-LU-1 (lung adenocarcinoma), HepG2 (hepatocellular carcinoma), PLC (pancreatic carcinoma), MCF-7 (breast adenocarcinoma), A549 (lung adenocarcinoma), BxPC3 (pancreatic adenocarcinoma), and LN229 (glioblastoma). Among them, U87-MG and BxPC3 were characterized as high PD-L1 presenting cell lines, while SK-LU-1, A549, and LN229 were categorized as middle PD-L1 presenting cell lines. The remaining cell lines, HepG2, PLC, and MCF-7, exhibited low expression levels of PD-L1.

FIG. 11 shows the cytotoxicity of TT0003 CAR-T cells across eight cancer cell lines, assessed using the CCK-8 assay. MDA-MB-231 PD-L1^{KO} served as the negative control. Co-culturing times of 24 or 48 hours and E:T ratios of 2 or 5 were employed for all tests. At 24 hours of co-culturing, PLC exhibited low cytotoxicity, while A549 and MCF-7 displayed moderate cytotoxicity. Conversely, the remaining cell lines demonstrated strong cytotoxic effects. By the 48-hour mark, only PLC retained moderate cytotoxicity, with the other cell lines exhibiting high cytotoxicity.

FIG. 12 shows the expansion and viability of CAR-T cells over a 12-day culturing period. The top panel illustrates that the AtezoCAR-T group exhibited the least expansion, with a fold increase of approximately 16, significantly lower than that of the TT0003 CAR-T group, which showed a 44-fold expansion, closely aligning with the control CD3+ T group's 62-fold expansion. The bottom panel details the viability of T cells during this expansion phase. It shows that the viability of AtezoCAR-T decreased to 80% after 12 days of culturing. In contrast, both TT0003 CAR-T and CD3+ T groups demonstrated an ability to restore cell viability to over 90% after viral transduction. The figure indicates stronger self-amplifying cytotoxic effects (fratricide) in the AtezoCAR-T compared to the other groups.

FIGS. 13A to 13F show screening of 2^{nd} generation CAR-designed gPD-L1 CAR NK cells. (FIG. 13A) The schematics depicting the modular structures of gPD-L1-CARs. The hinge for p280, p281, p285 and p286 is CD8 hinge while the hinge for p287 and p288 is CD28 hinge. (FIG. 13B) Summary of transduction rates (GFP %) of different gPD-L1 CAR-NK cells post transduction Day 6. Each data point (dot) represents one batch of gPD-L1 CAR-NK cell production. (FIG. 13C) Purities (CD56+ and CD3−) of different gPD-L1 CAR-NK cells. (FIG. 13D) Viable cell expansion folds of different gPD-L1 CAR-NK cells post transduction Day 6. (FIG. 13E) Representative images of a 3D-tumor spheroid cytotoxicity assay of MDA-MB-231_mcherry_Luc breast cancer cells. (FIG. 13F) Luciferase-based cytotoxicity assays of different gPD-L1 CAR-NK cells with MDA-MB-231_mcherry_Luc cancer cells.

FIGS. 14A to 14E. Production and characterization of gPD-L1 CAR-NK cells and Atezo CAR-NK cells. (FIG. 14A) Schematic representation of gPD-L1 CAR (p285) and Atezo CAR (p323). Antigen recognition domain: (anti-gPD-L1 scFv); hinge domain: CD8 Hinge; transmembrane domain: CD8TM); 4-1BB co-stimulatory domain; CD3ζ activation domain; reporter gene: EGFP. (FIG. 14B) Summary of transduction rates (GFP %) of gPD-L1 CAR-NK cells and Atezo CAR-NK cells post transduction Day 6. Each data point (dot) represents one batch of gPD-L1 CAR-NK cell production. (FIG. 14C) One batch representation for the purities (CD56⁺ and CD3⁻) of gPD-L1 CAR NK cells and Atezo CAR-NK cells. (FIG. 14D) One batch representation of viable cell expansion folds of gPD-L1 CAR-NK cells and Atezo CAR-NK cells. (FIG. 14E) Cytotoxicity assessment of gPD-L1 CAR-NK cells and Atezo CAR-NK cells against MDA-MB231 cells. Cytolytic activity of gPD-L1 CAR-NK cells against MDA-MB231-Luc cells at E/T ratios of 0.125, 0.25, 0.5, 1 and 2 after co-culture for 24 hrs was measured using luciferase-based reporter assay.

FIG. 15. Cytokine releasing profiles of gPD-L1 CAR-NK cells co-cultured with MDA-MB-231 cells. The supernatant of gPD-L1 CAR-NK cells co-cultured with MDA-MB-231-Luc cells at E/T ratio of 2 for 24 hrs was assayed for multiple cytokine secretion using LEGENDplex™ Human CD8/NK Panel.

FIGS. 16A to 16B show gPD-L1 CAR-NK cells mediated cytotoxicity against multiple cancer cell lines. (FIG. 16A) PD-L1 expression levels on various cancer cell lines. (FIG. 16B) Cytolytic activity of gPD-L1 CAR-NK cells and Atezo CAR-NK cells against multiple cancer cell lines. Multiple cancer cell lines were co-cultured with gPD-L1 CAR-NK cells and Atezo CAR-NK cells at different E/T ratios of 0.125, 0.25, 0.5, and 1 for 24 hrs and the cytolytic activity of each CAR-NK cell was measured by luciferase-based reporter assay.

FIGS. 17A to 17B show that gPD-L1 CAR-NK cells are less susceptible to macrophage interference. (FIG. 17A) Image-based analysis of CAR-NK cell-mediated cytotoxicity in the absence or presence of macrophages. Green: NK cells; Blue: Macrophage; Red: MDA-MB-231. (FIG. 17B) Cytolytic activity of each CAR-NK cell in the absence or presence of macrophages. Cytolytic activity of gPD-L1 CAR-NK cells and Atezo CAR-NK cells against MDA-MB231 at E/T ratios of 0.5, 1 and 2 in the absence or presence of macrophages (E/T=0.5) after co-culture for 24 hrs was measured by luciferase-based reporter assay.

FIG. 18 shows that under MOI 5 condition, both TT0003 CAR-T and two batches of AtezoCAR-T exhibited over 90% of cells being CAR⁺.

FIG. 19 shows that 50% of TT0003 CAR-T cells still retained PD-L1 expression, while only 2% and 4% of cells in the two batches of AtezoCAR-T were PD-L1⁺.

FIG. 20 shows cytotoxicity analysis in breast cancer cell line MDA-MB-231 GFP/Luc with CAR-T Treatment. FIG. 20A shows the cytotoxicity of AtezoCAR-T and TT0003 CAR-T cells against MDA-MB-231 breast cancer cells, measured using the Luciferase assay after 24 hours of co-culture. FIG. 20B shows the cytotoxicity of AtezoCAR-T and TT0003 CAR-T cells against MDA-MB-231 breast cancer cells, measured using the Luciferase assay after 48 hours of co-culture. In contrast, murine CAR-T cells required 48 hours to reach the level of cytotoxicity observed with TT0003 CAR-T cells at 24 hours.

FIG. 21 shows IFN-γ Release Specificity Ratio of AtezoCAR-T, TT0003 CAR-T, and murine CAR-T. TT0003 CAR-T exhibited the highest specificity for MDA-MB-231 tumor cells, demonstrating enhanced therapeutic potential with minimized on-target, off-tumor effects.

FIGS. 22A to 22B show the experimental results of TT0003 CAR-T cells in the MDA-MB-231 xenograft mouse model, with CAR-T administration performed on day 11. The negative control group was treated with DPBS. FIG. 22A illustrates the changes in tumor size over time, while FIG. 22B depicts the relative body weight changes of the mice during the experimental period.

FIG. 23 shows that gPD-L1 CAR-NK cells reduced tumor growth of orthotopic MDA-MB-231 xenografts. (Top panel) Bioluminescence images of orthotopic xenografted MDA-MB-231 fLuc(+) tumor growth in advanced severe immunodeficiency (ASID) mice before and after first NK or gPD-L1 CAR-NK cell peritumoral injection on Day 11. (Bottom panel) Tumor volumes of MDA-MB-231 tumors (n=5) treated with the control saline, mock NK cells (1×10⁷ cells/dose), or gPD-L1 CAR-NK cells (1×10⁷ cells/dose, ˜95% CAR⁺). Tumor dimensions were measured using calipers, and tumor volumes were calculated with the formula V=½(length×width²).

FIG. 24 shows the Biacore T200 analysis of the binding affinity between hTT-01 and PD-L1.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

### I. General definitions

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “approximately” or “about” refers to a degree of acceptable deviation that will be understood by persons of ordinary skill in the art, which may vary to some extent depending on the context in which it is used. Specifically, “approximately” or “about” may mean a numeric value having a range of ±10% or ±5% or ±3% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e. full-length) antibody molecules but also antigen-binding fragments thereof retaining antigen binding ability e.g. Fab, Fab′, F(ab′)2 and Fv. Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies) and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (V_{H}) and a first, second and third constant regions (C_{H}1, C_{H}2 and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contain three highly variable regions, called the complementarity determining regions (CDRs); namely, heavy (H) chain CDRs including HC CDR1, HC CDR2, HC CDR3 and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)2 fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) a (scFv)₂, which can contain two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammal (e.g., a non-human mammal such as mouse, rabbit and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences e.g. by mutations in one or more of the CDRs, or in one or more of the FRs, such as to, for example, decrease possible immunogenicity, increase affinity, and eliminate cysteines that might cause undesirable folding, etc.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specifically binding when used with respect to an antibody can refer to an antibody that specifically binds to (recognize) its target with an KD value less than about 10⁻⁷ M, such as about 10⁻⁸ M or less, such as about 10⁻⁹ M or less, about 10⁻¹⁰ M or less, about 10⁻¹¹ M or less, about 10⁻¹² M or less, or even less, and binds to the specific target with an affinity corresponding to a K_{D} that is at least ten-fold lower than its affinity for binding to a non-specific antigen (such as BSA or casein), such as at least 100 fold lower, e.g. at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-GATAT-3′ is complementary to a polynucleotide whose sequence is 5′-ATATC-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed.

Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has had introduced into it a recombinant nucleic acid. “A transformed cell” mean a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOXI) promoter), a start codon, a replication origin, enhancers, a secretion signal sequence (e.g., α-mating factor signal), a stop codon, and other control sequence (e.g., Shine-Dalgarno sequences and termination sequences). Preferably, vectors may further contain a marker sequence (e.g., an antibiotic resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification e.g. a His-tag.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

### II. Antibodies Against PD-L1

The present invention is based, at least in part, on antibodies against PD-L1,for preparing a chimeric antigen receptor (CAR). The anti-PD-L1 antibodies as used herein are found to specifically target PD-L1 at certain glycosylation sites. The anti-PD-L1 antibodies as used herein are useful in developing a new CAR construct aiming to improve the efficacy of CAR-T therapy and enhance safety e.g. reducing off-tumor toxicity.

U.S. Pat. No. 11,660,352 B2 describes certain murine anti-PD-L1 antibodies, the relevant disclosures of which are incorporated by reference herein for the purposes or subject matter referenced herein. An exemplary murine anti-PD-L1 antibody comprises a heavy chain variable region (V_{H}) having complementary determining regions thereof (HC CDR1, HC CDR2, and HC CDR3) and a light chain variable region (V_{L}) having complementary determining regions thereof (LC CDR1, LC CDR2, and LC CDR3) as shown in Table 1 below.

TABLE 1
Amino acid sequences of the murine
anti-PD-L1 antibody, mTT-01

| Anti-PD-L1 | |
|---|---|
| antibody, mTT-01 | Amino acid sequence |
| Heavy Chain CDR1 | NYVMS (SEQ ID NO: 1) |
| Heavy Chain CDR2 | TISSGGRYIYYTDSVKG (SEQ ID NO: 2) |
| Heavy Chain CDR3 | DGSTLYYFDY (SEQ ID NO: 3) |
| V_{H} Domain | EVMLVESGGALVKPGGSLKLSCAASGESLSNYVMS |
| | WVRQTPEKRLEWVATISSGGRYIYYTDSVKGRFTI |
| | SRDNARNTLYLQMSSLRSEDTAMYYCARDGSTLYY |
| | FDYWGQGTTLTVSS (SEQ ID NO: 7) |
| Light Chain CDR1 | SASSSVDYMY (SEQ ID NO: 4) |
| Light Chain CDR2 | DTSNLAS (SEQ ID NO: 5) |
| Light Chain CDR3 | QQWSSSPPIT (SEQ ID NO: 6) |
| V_{L} Domain | QTVLTQSPAIMSASPGEKVTMTCSASSSVDYMYWY |
| | QQKPGSSPRLLIYDTSNLASGVPVRESGSGSGTSY |
| | SLTISRMEAEDAATYYCQQWSSSPPITFGTGTKVE |
| | LK (SEQ ID NO: 8) |

TABLE 2
DNA sequences of the murine anti-PD-L1
antibody, mTT-01
Anti-PD-L1 antibody, mTT-01
V_{H} Domain
GAAGTGATGCTGGTGGAGTCTGGGGGAGCCTTAGTGAAGCCTGGAGGGTC
CCTGAAACTCTCCTGTGCAGCTTCTGGATTCAGTTTGAGTAACTATGTCA
TGTCTTGGGTTCGCCAGACTCCAGAGAAGAGGCTGGAGTGGGTCGCAACC
ATTAGTAGTGGTGGTAGGTATATCTACTATACAGACAGTGTGAAGGGTCG
ATTCACCATCTCCAGGGACAATGCCAGGAACACCCTGTACCTGCAAATGA
GCAGTCTGAGGTCTGAGGACACGGCCATGTATTATTGTGCAAGAGACGGT
AGTACCTTGTACTACTTTGACTATTGGGGCCAAGGCACCACTCTCACAGT
CTCCTCA (SEQ ID NO: 9)
V_{L} Domain
CAAACTGTTCTCACCCAGTCTCCAGCAATCATGTCTGCATCTCCAGGGGA
GAAGGTCACCATGACCTGCAGTGCCAGCTCAAGTGTAGATTACATGTACT
GGTACCAGCAGAAGCCAGGATCCTCCCCCAGACTCCTGATTTATGACACA
TCCAACCTGGCTTCTGGAGTCCCTGTTCGCTTCAGTGGCAGTGGGTCTGG
GACCTCTTACTCTCTCACAATCAGCCGAATGGAGGCTGAAGATGCTGCCA
CTTATTACTGCCAGCAGTGGAGTAGTTCCCCACCCATCACGTTCGGTACT
GGGACCAAGGTGGAGCTGAAA (SEQ ID NO: 10)

In the present invention, the anti-PD-L1 antibody as used herein is a humanized anti-PD-L1 antibody.

In some embodiments, an humanized anti-PD-L1 antibody of the present invention comprises a V_{H} having HC CDR1, HC CDR2, and HC CDR3, and a V_{L} having LC CDR1, LC CDR2, and LC CDR3 as shown in Table 3 below.

TABLE 3
Amino acid sequences of a humanized
anti-PD-L1 antibody, hTT-01.

| Anti-PD-L1 | |
|---|---|
| antibody, hTT-01 | Amino acid sequence |
| Heavy Chain CDR1 | NYVMS (SEQ ID NO: 1) |
| Heavy Chain CDR2 | TISSGGRYIYYTDSVKG (SEQ ID NO: 2) |
| Heavy Chain CDR3 | DGSTLYYFDY (SEQ ID NO: 3) |
| V_{H} Domain | EVQLVESGGGLVKPGGSLRLSCAASGFSLSNYVMS |
| | WVRQAPGKGLEWVATISSGGRYIYYTDSVKGRFTI |
| | SRDNAKNSLYLQMNSLRAEDTAVYYCARDGSTLYY |
| | FDYWGQGTTVTVSS (SEQ ID NO: 12) |
| Light Chain CDR1 | SASSSVDYMY (SEQ ID NO: 4) |
| Light Chain CDR2 | DTSNLAT (SEQ ID NO: 11) |
| Light Chain CDR3 | QQWSSSPPIT (SEQ ID NO: 6) |
| V_{L} Domain | QTVLTQSPATLSLSPGERATLSCSASSSVDYMYWY |
| | QQKPGQAPRLLIYDTSNLATGIPARFSGSGSGTDF |
| | TLTISSLEPEDFATYYCQQWSSSPPITFGQGTKVE |
| | IK (SEQ ID NO: 13) |

TABLE 4
DNA sequences of the humanized anti-PD-L1
antibody, hTT-01
Humanized Anti-PD-L1 antibody, hTT-01
V_{H} Domain
GAAGTGCAGCTCGTGGAGTCCGGTGGCGGACTCGTGAAACCTGGGGGAAG
CCTGCGGTTGTCATGTGCCGCTTCGGGGTTCAGCCTGTCCAACTACGTGA
TGTCGTGGGTCAGACAGGCGCCGGGAAAGGGACTTGAATGGGTGGCCACT
ATTAGCTCGGGCGGCAGATACATCTACTACACCGACTCCGTCAAGGGCCG
GTTTACCATCTCCCGCGACAACGCCAAGAACTCCCTGTATCTGCAAATGA
ACTCACTGCGGGCAGAGGATACCGCCGTGTACTACTGCGCCCGCGATGGT
TCCACCCTGTACTACTTCGACTACTGGGGACAGGGCACCACTGTGACGGT
GTCCTCA (SEQ ID NO: 14)
V_{L} Domain
CAGACCGTGCTGACTCAGAGCCCGGCCACACTTTCCCTGTCCCCGGGAGA
AAGGGCTACCTTGTCCTGCTCCGCGTCGTCCTCCGTGGACTATATGTACT
GGTACCAGCAGAAGCCAGGTCAAGCCCCTCGCCTGCTCATCTACGACACT
TCCAACCTCGCCACCGGAATCCCTGCCCGGTTCAGCGGATCAGGCTCCGG
CACCGACTTTACCCTGACCATTTCCTCGCTGGAGCCCGAGGATTTCGCAA
CCTACTACTGTCAGCAATGGTCAAGCTCGCCCCCGATCACGTTCGGCCAG
GGGACTAAGGTCGAAATCAAA (SEQ ID NO: 15)

In some embodiments, the anti-PD-L1 antibody is a functional variant of hTT-01 which is characterized in comprising (a) a V_{H} comprising HC CDR1 of SEQ ID NO: 1, HC CDR2 of SEQ ID NO: 2, and HC CDR3 of SEQ ID NO: 3; and (b) a V_{L} comprising LC CDR1 of SEQ ID NO: 4, LC CDR2 of SEQ ID NO: 11, and HC CDR3 of SEQ ID NO: 6. In some embodiments, the anti-PD-L1 antibody can comprise a V_{H} comprising SEQ ID NO: 12 or an amino acid sequence substantially identical thereto and a V_{L} comprising SEQ ID NO: 13 or an amino acid sequence substantially identical thereto. Specifically, the anti-PD-L1 antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO:12, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO:13. The anti-PD-L1 antibody of the present invention also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g., Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

Antigen-binding fragments of the antibodies described herein include a Fab fragment, a F(ab′)2 fragment, a Fv fragment, a single chain Fv (scFv) and a (scFv)₂. The antibodies or their antigen-binding fragments can be prepared by methods known in the art. The antibodies described herein may be animal antibodies (e.g., mouse-derived antibodies), chimeric antibodies (e.g., mouse-human chimeric antibodies), humanized antibodies, or human antibodies.

### III. Preparation of Antibodies or Antigen-Binding Fragments Thereof

Numerous methods conventional in this art are available for obtaining antibodies or antigen-binding fragments thereof.

In some embodiments, the antibodies provided herein may be made by the conventional hybridoma technology. In general, a target antigen optionally coupled to a carrier protein, e.g. keyhole limpet hemocyanin (KLH), and/or mixed with an adjuvant, e.g. complete Freund's adjuvant, may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of nucleotide sequences encoding the heavy and light chain variable regions of the antibodies as described herein are as shown in Table 4. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of an full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

### IV. Chimeric Antigen Receptor (CARs)

A chimeric antigen receptor (CAR) is an artificial immune cell receptor that allow the T cells to recognize a particular antigen of a targeted cell (e.g. a tumor cell). In general, a CAR is a fusion polypeptide comprising an antigen-binding extracellular domain that recognizes a target antigen, a transmembrane domain and an intracellular signaling domain. A T cell that expresses a CAR polypeptide is referred to as a CAR T cell that can specifically target certain antigens of interest.

In some embodiments, a CAR can include a signal peptide at the N-terminus. A signal peptide includes a peptide sequence that directs the transport and localization of the peptide within a cell and/or the cell surface. In one embodiment, the signal peptide includes the signal peptide from human CD8a (MALPVTALLLPLALLLHAARP) (SEQ ID NO: 16). In another embodiment, the signal peptide includes the signal peptide from human CD8b (MRPRLWLLLAAQLTVLHGNSV) (SEQ ID NO: 17). Other examples include the signal peptide from human CD45 and interleukin-2 (IL-2). Functional equivalents are included which are, for example, CD8a signal peptides, CD8b signal peptides, CD45 signal peptides and IL-2 signal peptides from homologous proteins from other species. Signal peptides may cleave either during or after translocation to generate a free signal peptide and a mature protein.

The antigen-binding extracellular domain is the region of a CAR polypeptide exposed to the extracellular fluid when the CAR is expressed on cell surface. Typically, the antigen-binding extracellular domain is a single-chain variable fragment (scFv) derived from a monoclonal antibody while it can be based on other formats which comprise an antibody-like antigen binding site. A scFv may include an antibody heavy chain variable region (V_{H}) and an antibody light chain variable region (V_{L}), having a V_{H}-V_{L} or a V_{L}-V_{H} orientation. In some embodiments, the antigen-binding extracellular domain is a scFv derived from anti-PD-L1 antibodies as described herein e.g. hTT-01. In some embodiments, the V_{H} and V_{L} may be linked to each other via a peptide linker. The peptide linker may be 5-25 amino acid residues in length, 25-100 amino acid residues in length, or 50-200 amino acid residues in length. In some embodiments, the peptide linker is a Gly-Ser linker. In a particular example, a Gly-Ser linker includes the amino acid sequence of GGGGSGGGGSGGGGS (SEQ ID NO: 18). In some embodiments, the antigen-binding extracellular domain includes, from N-terminus to C-terminus, hTT-01 V_{H}, a linker and hTT-01 V_{L}. In a particular example, the antigen-binding extracellular domain contains the amino acid sequence set forth in SEQ ID NO: 19 In some embodiments, the antigen-binding extracellular domain includes, from N-terminus to C-terminus, hTT-01 V_{L}, a linker and hTT-01 V_{H}. In a particular example, the antigen-binding extracellular domain contains the amino acid sequence set forth in SEQ ID NO: 20

TABLE 5
Amino acid sequences of antigen-binding
extracellular domains based on hTT-01.
hTT-01 V_{H}+ linker+ hTT-01 V_{L}
EVQLVESGGGLVKPGGSLRLSCAASGESLSNYVMSWVRQAPGKGLEWVAT
ISSGGRYIYYTDSVKGRFTISRDNAKNSLYLQMNSLRAEDTAVYYCARDG
STLYYFDYWGQGTTVTVSSGGGGSGGGGSGGGGSQTVLTQSPATLSLSPG
ERATLSCSASSSVDYMYWYQQKPGQAPRLLIYDTSNLATGIPARFSGSGS
GTDFTLTISSLEPEDFATYYCQQWSSSPPITFGQGTKVEIK (SEQ ID
NO: 19)
hTT-01 V_{L}+ linker+ hTT-01 V_{H}
QTVLTQSPATLSLSPGERATLSCSASSSVDYMYWYQQKPGQAPRLLIYDT
SNLATGIPARFSGSGSGTDFTLTISSLEPEDFATYYCQQWSSSPPITFGQ
GTKVEIKGGGGSGGGGSGGGGSEVQLVESGGGLVKPGGSLRLSCAASGFS
LSNYVMSWVRQAPGKGLEWVATISSGGRYIYYTDSVKGRFTISRDNAKNS
LYLQMNSLRAEDTAVYYCARDGSTLYYFDYWGQGTTVTVSS (SEQ ID
NO: 20)

The CAR polypeptide described herein may contain a transmembrane domain which is typically an alpha helix comprising several hydrophobic residues that spans the cell membrane. The transmembrane domain can provide stability of the CAR polypeptide containing it. In some embodiments, the transmembrane domain can be a CD28 transmembrane domain, a CD8 transmembrane domain, or a chimera of a CD8 and CD28 transmembrane domain. In some embodiments, the transmembrane domain is a CD8a transmembrane domain containing the sequence of:

| (SEQ ID NO: 21) | |
|---|---|
| | IWAPLAGTCGVLLLSLVITLYC. |

The CAR polypeptide described herein may contain a hinge domain located between the transmembrane domain and the antigen binding domain. In some embodiments, a hinge domain may comprise up to 300 amino acids e.g. 5 to 20 amino acids, 15 to 50 amino acids, 20 to 100 amino acids or 30 to 200 amino acids. A hinge domain may provide flexibility to the CAR, or to prevent steric hindrance of the CAR. In some embodiments, the hinge domain is a CD8 hinge domain. In a preferred embodiment, the CD8 hinge domain is human. In some embodiments, the hinge domain is a CD28 hinge domain. In a preferred embodiment, the CD28 hinge domain is human. In some embodiments, the hinge domain is a CD8a hinge domain containing the sequence of:

(SEQ ID NO: 22)

TTTPAPRPPTPAPTIASQPLSLRPEACRPAAGGAVHTRGLDFACDIY.

The intracellular signaling domain of a CAR polypeptide is capable of activating at least one of the normal effector functions of the immune cell engineered to express the CAR polypeptide. Effector function of a T cell, for example, may be cytolytic activity or helper activity including the secretion of cytokines. The intracellular signaling domain of a CAR polypeptide can be a portion of a protein that transduces the effector function signal and directs the cell to perform a specialized function. In particular, the intracellular signaling domain is derived from the intracellular signaling domain of a native receptor. Examples of such native receptors include the zeta (ζ) chain of the T-cell receptor or any of its homologs (e.g., delta, gamma, or epsilon). For example, CD3ζ (CD3-zeta) is the cytoplasmic signaling domain of the CD3 complex of the T cell receptor (TCR). It contains three immunoreceptor tyrosine-based activation motifs (ITAMs) that activate downstream signaling pathways. In some embodiments, the intracellular signaling domain include a CD3-zeta signaling domain containing the sequence of: RVKFSRSADAPAYKQGQNQLYNELNLGRREEYDVLDKRRGRDPEMGGKPRR KNPQEGLYNELQKDKMAEAYSEIGMKGERRRGKGHDGLYQGLSTATKDTYD ALHMQALPPR (SEQ ID NO: 23). CD3-zeta may provide a primary T cell activation signal but not a fully competent activation signal and thus additional co-stimulatory signaling may be needed. A costimulatory molecule is a cell surface molecule other than an antigen receptor or its ligands that is required for an efficient response of lymphocytes to an antigen. Examples of costimulatory molecules include but not limited to CD27, CD28, 4-1BB (CD137), OX40, CD30, lymphocyte function-associated antigen-1 (LFA-1) and CD2. In some instances, the co-stimulatory domains of CD28 and/or 4-1BB may be used to transmit a full proliferative/survival signal, together with the primary signaling mediated by CD3ζ. In some embodiments, a CAR polypeptide disclosed herein comprises a CD28 co-stimulatory molecule. In some embodiments, a CAR polypeptide disclosed herein comprises a 4-1BB co-stimulatory molecule. In some embodiments, the co-stimulatory molecule includes a 4-1BB co-stimulatory molecule containing the sequence of: KRGRKKLLYIFKQPFMRPVQTTQEEDGCSCRFPEEEEGGCEL (SEQ ID NO: 24). In some embodiments, a CAR polypeptide disclosed herein comprises a CD3ζ signaling domain with a CD28 co-stimulatory domain. In some embodiments, a CAR polypeptide disclosed herein comprises a CD35 signaling domain with a 4-1BB co-stimulatory domain. In still other embodiments, a CAR includes a CD3ζ signaling domain with a CD28 co-stimulatory domain and a 4-1BB co-stimulatory domain.

In one particular embodiment, a CAR polypeptide disclosed herein comprises the amino acid sequence as set forth in SEQ ID NO: 25 (TT0003: V_{L}-linker-V_{H} chain+Ala-Ala-Ala+CD8a hinge+CD8a transmembrane domain+4-1BB+CD3-zeta). In another particular embodiments, a CAR polypeptide disclosed herein comprises the amino acid sequence as set forth in SEQ ID NO: 26 (TT0004: V_{H}-linker-V_{L} chain+Ala-Ala-Ala+CD8a hinge+CD8a transmembrane domain+4-1BB+CD3-zeta).

TABLE 6
Amino acid sequences of a CAR polypeptide
disclosed herein based on hTT-01.
TT0003: V_{L}-linker-V_{H} chain+Ala-Ala-Ala+CD8a
hinge+CD8a transmembrane domain+4-1BB+CD3-zeta
QTVLTQSPATLSLSPGERATLSCSASSSVDYMYWYQQKPGQAPRLLIYDT
SNLATGIPARFSGSGSGTDFTLTISSLEPEDFATYYCQQWSSSPPITFGQ
GTKVEIKGGGGSGGGGSGGGGSEVQLVESGGGLVKPGGSLRLSCAASGFS
LSNYVMSWVRQAPGKGLEWVATISSGGRYIYYTDSVKGRFTISRDNAKNS
LYLQMNSLRAEDTAVYYCARDGSTLYYFDYWGQGTTVTVSSAAATTTPAP
RPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEE
GGCELRVKFSRSADAPAYKQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR (SEQ ID NO: 25)
TT0004: V_{H}-linker-V_{L} chain+Ala-Ala-Ala+CD8a
hinge+CD8a transmembrane domain+4-1BB+CD3-zeta
EVQLVESGGGLVKPGGSLRLSCAASGFSLSNYVMSWVRQAPGKGLEWVAT
ISSGGRYIYYTDSVKGRFTISRDNAKNSLYLQMNSLRAEDTAVYYCARDG
STLYYFDYWGQGTTVTVSSGGGGSGGGGSGGGGSQTVLTQSPATLSLSPG
ERATLSCSASSSVDYMYWYQQKPGQAPRLLIYDTSNLATGIPARFSGSGS
GTDFTLTISSLEPEDFATYYCQQWSSSPPITFGQGTKVEIKAAATTTPAP
RPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEE
GGCELRVKESRSADAPAYKQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR (SEQ ID NO: 26)
P280
Signal sequence
MLLLVTSLLLCELPHPAFLLIP (SEQ ID NO: 27)
ScFv
EVQLVESGGGLVKPGGSLRLSCAASGFSLSNYVMSWVRQAPGKGLEWVAT
ISSGGRYIYYTDSVKGRFTISRDNAKNSLYLQMNSLRAEDTAVYYCARDG
STLYYFDYWGQGTTVTVSSGGGGSGGGGSGGGGSQTVLTQSPATLSLSPG
ERATLSCSASSSVDYMYWYQQKPGQAPRLLIYDTSNLATGIPARFSGSGS
GTDFTLTISSLEPEDFATYYCQQWSSSPPITFGQGTKVEIK (SEQ ID
NO: 19)
Hinge
TTTPAPRPPTPAPTIASOPLSLRPEACRPAAGGAVHTRGLDFACD (SEQ
ID NO: 28)
Transmembrane domain (TM)
PFFFCCFIAVAMGIRFIIMVT (SEQ ID NO: 29)
Stimulation domain
WRRKRKEKQSETSPKEFLTIYEDVKDLKTRRNHEQEQTFPGGGSTIYSMI
QSQSSAPTSQEPAYTLYSLIQPSRKSGSRKRNHSPSENSTIYEVIGKSQP
KAQNPARLSRKELENEDVYS (SEQ ID NO: 30)
Co-stimulation domain
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO: 23)
P281
Signal sequence
MLLLVTSLLLCELPHPAFLLIP (SEQ ID NO: 27)
ScFv
QTVLTQSPATLSLSPGERATLSCSASSSVDYMYWYQQKPGQAPRLLIYDT
SNLATGIPARFSGSGSGTDFTLTISSLEPEDFATYYCQQWSSSPPITFGQ
GTKVEIKGGGGSGGGGSGGGGSEVQLVESGGGLVKPGGSLRLSCAASGFS
LSNYVMSWVRQAPGKGLEWVATISSGGRYIYYTDSVKGRFTISRDNAKNS
LYLQMNSLRAEDTAVYYCARDGSTLYYFDYWGQGTTVTVSS (SEQ ID
NO: 20)
Hinge
TTTPAPRPPTPAPTIASQPLSLRPEACRPAAGGAVHTRGLDFACD (SEQ
ID NO: 28)
Transmembrane domain (TM)
PFFFCCFIAVAMGIRFIIMVT (SEQ ID NO: 29)
Stimulation domain
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENEDVYS (SEQ ID NO: 30)
Co-stimulation domain
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO: 23)
P285
Signal sequence
MLLLVTSLLLCELPHPAFLLIP (SEQ ID NO: 27)
ScFv
EVQLVESGGGLVKPGGSLRLSCAASGFSLSNYVMSWVRQAPGKGLEWVAT
ISSGGRYIYYTDSVKGRFTISRDNAKNSLYLQMNSLRAEDTAVYYCARDG
STLYYFDYWGQGTTVTVSSGGGGSGGGGSGGGGSQTVLTQSPATLSLSPG
ERATLSCSASSSVDYMYWYQQKPGQAPRLLIYDTSNLATGIPARFSGSGS
GTDFTLTISSLEPEDFATYYCQQWSSSPPITEGQGTKVEIK (SEQ ID
NO: 19)
Hinge
TTTPAPRPPTPAPTIASQPLSLRPEACRPAAGGAVHTRGLDFACD (SEQ
ID NO: 28)
Transmembrane domain (TM)
IYIWAPLAGTCGVLLLSLVITLYC (SEQ ID NO: 31)
Stimulation domain
KRGRKKLLYIFKQPFMRPVQTTQEEDGCSCRFPEEEEGGCEL (SEQ ID
NO: 24)
Co-stimulation domain
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO: 23)
P286
Signal sequence
MLLLVTSLLLCELPHPAFLLIP (SEQ ID NO: 27)
ScFv
QTVLTQSPATLSLSPGERATLSCSASSSVDYMYWYQQKPGQAPRLLIYDT
SNLATGIPARFSGSGSGTDFTLTISSLEPEDFATYYCQQWSSSPPITFGQ
GTKVEIKGGGGSGGGGSGGGGSEVQLVESGGGLVKPGGSLRLSCAASGFS
LSNYVMSWVRQAPGKGLEWVATISSGGRYIYYTDSVKGRFTISRDNAKNS
LYLQMNSLRAEDTAVYYCARDGSTLYYFDYWGQGTTVTVSS (SEQ ID
NO: 20)
Hinge
TTTPAPRPPTPAPTIASQPLSLRPEACRPAAGGAVHTRGLDFACD (SEQ
ID NO: 28)
Transmembrane domain (TM)
IYIWAPLAGTCGVLLLSLVITLYC (SEQ ID NO: 31)
Stimulation domain
KRGRKKLLYIFKQPFMRPVQTTQEEDGCSCRFPEEEEGGCEL (SEQ ID
NO: 24)
Co-stimulation domain
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO: 23)
P287
Signal sequence
MLLLVTSLLLCELPHPAFLLIP (SEQ ID NO: 27)
ScFv
EVQLVESGGGLVKPGGSLRLSCAASGFSLSNYVMSWVRQAPGKGLEWVAT
ISSGGRYIYYTDSVKGRFTISRDNAKNSLYLQMNSLRAEDTAVYYCARDG
STLYYFDYWGQGTTVTVSSGGGGSGGGGSGGGGSQTVLTQSPATLSLSPG
ERATLSCSASSSVDYMYWYQQKPGQAPRLLIYDTSNLATGIPARFSGSGS
GTDFTLTISSLEPEDFATYYCQQWSSSPPITFGQGTKVEIK (SEQ ID
NO: 19)
Hinge
IEVMYPPPYLDNEKSNGTIIHVKGKHLCPSPLFPGPSKP (SEQ ID
NO: 32)
Transmembrane domain (TM)
FWVLVVVGGVLACYSLLVTVAFIIFWV (SEQ ID NO: 33)
Stimulation domain
RSKRSRLLHSDYMNMTPRRPGPTRKHYQPYAPPRDFAAYRS (SEQ ID
NO: 34)
Co-stimulation domain
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO: 23)
P288
Signal sequence
MLLLVTSLLLCELPHPAFLLIP (SEQ ID NO: 27)
ScFv
QTVLTQSPATLSLSPGERATLSCSASSSVDYMYWYQQKPGQAPRLLIYDT
SNLATGIPARFSGSGSGTDFTLTISSLEPEDFATYYCQQWSSSPPITFGQ
GTKVEIKGGGGSGGGGSGGGGSEVQLVESGGGLVKPGGSLRLSCAASGFS
LSNYVMSWVRQAPGKGLEWVATISSGGRYIYYTDSVKGRFTISRDNAKNS
LYLQMNSLRAEDTAVYYCARDGSTLYYFDYWGQGTTVTVSS (SEQ ID
NO: 20)
Hinge
IEVMYPPPYLDNEKSNGTIIHVKGKHLCPSPLFPGPSKP (SEQ ID
NO: 32)
Transmembrane domain (TM)
FWVLVVVGGVLACYSLLVTVAFIIFWV (SEQ ID NO: 33)
Stimulation domain
RSKRSRLLHSDYMNMTPRRPGPTRKHYQPYAPPRDFAAYRS (SEQ ID
NO: 34)
Co-stimulation domain
RVKFSRSADAPAYKOGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO: 23)

### V. Nucleic Acid (Vector) and CAR Expressing Cell

A nucleic acid can be provided which encodes a CAR as described herein. The nucleic acid sequence may be, for example, a DNA, an RNA or a cDNA sequence. A nucleic acid encoding a CAR can be inserted into a vector. The vector may be a plasmid or a viral vector. The vector may be capable of transfecting or transducing a T cell. For example, a viral vector can be used such as a retrovirus vector (e.g. an oncoretrovirus vector, a lentivirus vector, and a pseudotyped vector), an adenovirus vector, an adeno-associated virus (AAV) vector, a simian virus vector, a vaccinia virus vector or a sendai virus vector, an Epstein-Barr virus (EBV) vector, and a herpes simplex virus (HSV) vector.

A nucleic acid encoding a CAR can be introduced into a cell. In some embodiments, a nucleic acid encoding a CAR can be designed to insert into a genomic site of interest in the host T cells. In some embodiments, the target genomic site can be in a safe harbor locus. Specifically, a nucleic acid encoding a CAR can insert into a location within a TRAC gene to disrupt the TRAC gene in the genetically engineered T cells and express the CAR polypeptide. Disruption of TRAC results in loss of function of the endogenous TCR. A viral vector such as an AAV vector or a LV vector, which encodes a CAR polypeptide described herein may be incubated with T cells for a suitable period to allow for entry of the viral vector into the T cells. After transduction, the T cells may be cultured in a suitable cell culture medium for a suitable period for recovery. The genetically engineered T cells may be expanded in vitro under suitable conditions to produce a population of genetically engineered T cells as desired.

The cell used as in the genetic engineering process for expressing a CAR is not particularly limited. The cell may be any eukaryotic cell capable of expressing a CAR at the cell surface, e.g. an immunological cell. In particular, the cell may be an immune effector cell such as a T cell. The T cell may include helper T cells (TH cells), cytotoxicity T cells (CTLs), memory T cells and regulatory T cells (Treg cells).

In some embodiments, the cells used herein for expressing a CAR is natural killer (NK) cells. In comparison to the clinical side-effects associated with CAR-T cells, CAR-NK cells emerge as a safer alternative with superior therapeutic effects. Firstly, activated NK cells predominantly produce IFN-γ and GM-CSF, whereas CAR-T cells secret cytokines such as IL-1, IL-2, IL-6, TNF-a, IL-8, IL-10, and IL-15, which are strongly linked to cytokine release syndrome and neurotoxicity. Phase I/II trials have demonstrated that allogeneic NK cell infusions are well-tolerated, causing no graft-versus-host diseases or significant toxicities. Secondly, CAR-NK cells exhibit spontaneous killing of tumor cells by recognizing diverse ligands through various activating receptors. Thirdly, NK cells are abundantly available from various sources such as umbilical cord blood, peripheral blood, human embryonic stem cells (hESCs), induced pluripotent stem cells (iPSCs), and even NK-92 cell lines. Thus, CAR-NK cells combine the antigen specific targeting of CAR-T cells with the innate activity of NK cells creating a platform which provides the potential for ‘off-the-shelf’ application with a safer adverse effect profile. In the absence of an ideal CAR target antigen, NK cells can confer the well-defined innate and alloreactive potential against tumor cells irrespective of the antigen engaged. A dual targeting solution is achievable based simply on NK ADCC in the presence of antigen-specific monoclonal antibodies.

The cells may be from a sample isolated from a patient, a related or unrelated haematopoietic transplant donor or a completely unconnected donor, from cord blood, differentiated from an embryonic cell line or an inducible progenitor cell line, or derived from a transformed cell line. In some embodiments, CAR expressing cells may be created from a peripheral blood mononuclear cell (PBMC) which may be obtained from a patient's own peripheral blood, or a haematopoietic stem cell transplant from donor peripheral blood. The CAR expressing cells as produced may be further expanded in vitro under suitable conditions to produce a population of CAR expressing cells to an amount as needed e.g. a clinically relevant scale. The CAR expressing cells as produced as described herein may be harvested for therapeutic uses.

### VI. Pharmaceutical Composition

The present invention also relates to a pharmaceutical composition comprising a CAR expressing cell as described herein. The CAR expressing cell may be formulated with a pharmaceutically acceptable carrier for purpose of delivery. The present invention further relates to a pharmaceutical composition comprising an antibody against PD-L1 as described herein. The anti-PD-L1 antibody may be formulated with a pharmaceutically acceptable carrier for purpose of delivery. As used herein, “pharmaceutically acceptable” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising a CAR expressing cell or an anti-PD-L1 antibody as described herein as an active ingredient can be in a form of a solution such as an aqueous solution e.g. a saline solution. Appropriate excipients also include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The composition of the present invention may be delivered via a physiologically acceptable route, typically intravenous infusion.

### VII. Treatment

A population of genetically engineered CAR expressing cells or an antibody as described herein may be administered to a subject for therapeutic purpose. In particular, the present invention provides a method for treating a disease a subject having a disease, disorder or condition associated with an elevated expression of a tumor antigen comprising administering to the subject an effective amount of a cell genetically modified to express a CAR or an antibody as described herein. The present invention also provides use of a cell genetically modified to express a CAR or an antibody as described herein for manufacturing a medicament for treating a disease a subject having a disease, disorder or condition associated with an elevated expression of a tumor antigen. The present invention also provides a cell genetically modified to express a CAR or an antibody as described herein for use in treating a disease a subject having a disease, disorder or condition associated with an elevated expression of a tumor antigen.

The term “effective amount” used herein refers to the amount of an active ingredient to confer a desired biological effect in a treated subject. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience. In some examples, the effective amount of an active ingredient is to provide an anti-tumor effect such as reducing tumor size as compared with that without administration of the active ingredient. In some examples, the effective amount of an active ingredient is to provide an anti-tumor effect with a reduced side effect e.g. a reduced toxicity to non-target cells. In some embodiments, an effective amount of a genetically engineered cell population may comprise 10⁵ to 10⁷ cells, such as 1×10⁵ cells, 2×10⁵ cells, 3×10⁵ cells, 4×10⁵ cells, 5×10⁵ cells, 6×10⁵ cells, 7×10⁵ cells, 8×10⁵ cells, 9×10⁵ cells, 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 1×10⁷ cells, 2×10⁷ cells, 3×10⁷ cells, 4×10⁷ cells, 5×10⁷ cells, 6×10⁷ cells, 7×10⁷ cells, 8×10⁷ cells, 9×10⁷ cells, or multiples thereof, per kilogram of body weight.

A subject to be treated by the method of treatment as described herein can be a mammal, more preferably a human. Mammals include, but are not limited to, farm animals, sport animals, pets, primates, horses, dogs, cats, mice, and rats. A human subject who needs the treatment may be a human patient having, at risk for, or suspected of having a target disease/disorder. A subject suspected of having any of such target disease/disorder might show one or more symptoms of the disease/disorder. A subject at risk for the disease/disorder can be a subject having one or more of the risk factors for that disease/disorder.

In some embodiments, the subject has been determined to have a relatively higher level of a tumor antigen as compared to a reference level.

In some embodiments, the method may include measuring a tumor antigen in a tumor sample from a patient and comparing the level of the tumor antigen in the sample with a reference level. In some embodiments, based on the comparing, a patient determined to have an enhanced level of the tumor antigen is selected. Specifically, for example, an enhanced level can be higher than a reference level by more than 5%, 10%, 20%, 30%, 40%, 50%, 60%, 70%, 80%, 90% or 100% or more. A reference level with respect to a tumor antigen as described herein can refer to a level measured in control samples (e.g. tissues or cells or any biological sample free of cancer of an individual or from a population of normal individuals). The measurement can be performed using conventional detection and statistic methods. In some embodiments, the tumor antigen is PD-L1.

In some embodiments, the subject is suffered from cancer. In particular, the cancer is PD-L1 associated cancer which is relevant to PD-L1 gene or gene product expression or overexpression. Non-limiting examples of cancers that may be treated using a population of genetically engineered described herein include, but are not limited to, breast cancer, colon cancer, head and neck cancer, thyroid cancer, lung cancer, ovarian cancer, pancreatic cancer, gastric cancer, kidney cancer, glioblastoma and leukemia.

Administering may include transplantation of the genetically engineered CAR expressing cells or an antibody as described herein by a method or route such that a desired amount of the genetically engineered CAR expressing cells or the antibody delivered to and located at a desired site, such as a tumor site, leading to a desired therapeutic effect(s). For example, in some instances, an effective amount of the genetically engineered CAR expressing cells or an antibody as described herein can be administered via a systemic route of administration, such as injection and infusion. Injection includes, without limitation, intravenous, intramuscular, intraarterial, intrathecal, intracardiac, intraperitoneal, subcutaneous. In some particular embodiments, the route is intravenous.

It is known that cell fratricide may occur to CAR-T due to the same antigen being expressed on both cancer cells and CAR T cells, which causes loss of therapeutic efficacy and also reduce clinical benefits. In the case of CAR-T cells, fratricide-resistance can mean inhibition of cell-death induction including suppression of killing of neighboring cells (fratricide) or cell autonomous death (suicide). Surprisingly, the CAR cells of the present invention exhibit superior fratricide-resistance and expansion capacity and still keep effective in killing cancer cells. In some embodiments, CAR-T cells of the present invention exhibit comparable cell viability compared to normal T cells (without CAR modification), during cell culture under the same condition. For example, CAR-T cells of the present invention exhibit decrease of cell numbers of less than 15%, 10%, 5%, 3% or 1% when compared to normal T cells (without CAR modification), during cell culture under the same condition. In some instances, CAR-T cells of the present invention exhibit cell viability of 80% or higher, 85% or higher, 90% or higher, 95% or higher, 97% or higher, or 99% or higher during cell culture. In some embodiments, CAR-T cells of the present invention exhibit comparable cell expansion fold compared to normal T cells (without CAR modification), during cell culture under the same condition. For example, CAR-T cells of the present invention exhibit cell expansion fold of less than 30%, 25%, 20%, 10%, 5%, 3% or 1% decrease when compared to normal T cells (without CAR modification), during cell culture under the same condition. In some instances, CAR-T cells of the present invention exhibit cell expansion fold of 20-fold or higher, 25-fold or higher, 30-fold or higher, 35-fold or higher, 40-fold or higher, 45-fold or higher during cell culture. In some embodiments, the CAR-T cells of the present invention are of a substantial percentage expressing PD-L1, particularly, 10% or more, 15% or more, 20% or more, 25% or more, 30% or more, 35% or more, 40% or more, 45% or more, 50% or more of the CAR-T cells of the present invention being PD-L1 positive. In some embodiments, the CAR-T cells of the present invention are of a substantial percentage expressing PD-L1, particularly, 10% to 50%, 15% to 50%, 25% to 50%, 30% to 50%, 35% to 50%, 40% to 50% of the CAR-T cells of the present invention being PD-L1 positive.

In addition, the CAR cells of the present invention exhibit selective potency or immunity toward cancer cells such that the cells can be administered in an amount effective in treating cancer with less damage to normal cells. It is demonstrated in the present invention that the CAR cells as described herein induce selectively higher secretion of interferon-gamma (IFN-γ) against cancer cells than against normal cells. The IFN-γ release specificity can be assayed by measuring a level of IFN-γ secretion obtained from co-culture of the CAR cells of the present invention with cancer cells (a first level of IFN-γ secretion) and a level of IFN-γ secretion from co-culture of the CAR cells of the present invention with normal cells (a second level of IFN-γ secretion) and dividing the first level of IFN-γ secretion by the second level of IFN-γ secretion to obtain an IFN-γ release specificity ratio where an higher IFN-γ release specificity ratio is indicative of a higher potency or immunity against cancer cells. In some embodiments, the CAR cells of the present invention provide an IFN-γ release specificity ratio of 10 or higher, for example, 15 or higher, 20 or higher, 25 or higher, 30 or higher, 35 or higher or 40 or higher. In some examples, the CAR cells of the present invention provide an IFN-γ release specificity ratio ranging from 10 to 50, for example, 15 to 50, 20 to 50, 25 to 50, 30 to 50, 35 to 50 or 40 to 50. In some embodiments, an effective amount of the CAR cells of the present invention as an active ingredient for treating cancer is an amount of such ingredient that can inhibit growth of cancer cells. Specifically, such amount is effective in reducing the number of target cancer cells by at least 10%, e.g. 20%, 30%, 40%, 50%, 60%, 70%, 80%, 90%, or 100%, as compared with the number of target cancer cells without treatment with such ingredient. Preferably, such amount is selectively cytotoxic to target cancer cells. In some embodiments, the ingredient is administered in an amount that selectively provides more cytotoxicity to target cancer cells than to normal cells. In certain examples, the ingredient can be administered in such amount which is effectively in reducing the number of target cancer cells by more than 50% (e.g, 60%, 70%, 80%, 90% or 100%) when compared with the number of target cancer cells without treatment with the ingredient, while causing less cytotoxicity to normal cells e.g. reducing the number of normal cells by less than 50% (e.g. 40%, 30%, 20%, 10%, or less) when compared with the number of normal cells without treatment with the ingredient. In some embodiments, cancer cells and normal cells as described herein are human cells.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### 1. Material and Methods

### 1.1 Anti-PD-L1 Monoclonal Antibody

A murine anti-PD-L1 monoclonal antibody mTT-01 was prepared as described in U.S. Pat. No. 11,660,352 B2 having a V_{H} domain of SEQ ID NO: 7 and a V_{L} domain of SEQ ID NO: 8, as provided in Table 1. A humanized anti-PD-L1 monoclonal antibody hTT-01 was prepared from the murine anti-PD-L1 monoclonal antibody mTT-01. The humanized anti-PD-L1 monoclonal antibody hTT-01 has a V_{H} domain of SEQ ID NO: 12 and a V_{L} domain of SEQ ID NO: 13, as provided in Table 3. Chimeric-atezolizumab (anti-hPD-L1-mIgG1) was purchased from InvivoGen (Anti-hPD-L1-mIgG1 InvivoFit, Cat #: hpdl1-mab9-1), which is a recombinant monoclonal antibody that features the constant region of a mouse IgG1 isotype and the variable region of Atezolizumab.

### 1.2 Immunohistochemistry Assay for Human Tissues

For the immunohistochemical analysis, the following modified protocol was utilized. Slides, containing either formalin-fixed paraffin-embedded (FFPE) tissue or tissue microarrays (TMAs) sourced from SCMH (Show Chwan Memorial Hospital), were initially prepared by incubating at 60-65° C. (for FFPE tissue) or 70° C. (for TMAs) for 30 minutes. Deparaffinization was achieved through two consecutive 10-minute immersions in Surgipath Xylene (3803665, Leica). The slides were then rehydrated through a graded series of Surgipath Reagent Alcohol 100% (3803686, Leica), decreasing in concentration from 100%, 95%, 80%, to 70%, each step lasting 5 minutes, followed by a rinse in tap water.

Antigen retrieval was performed using Citrate-based Antigen Retrieval Solution, pH 6 (S2369, DAKO), suitable for PD-L1, in a pressure cooker for 30 minutes. After cooling to room temperature, sections were washed in Tris-buffered saline with Tween 20 (TBST; TBT999, Scytek) for 5 minutes. Endogenous peroxidase activity was blocked by incubating sections in Hydrogen Peroxide Blocking Solution (TA-060-HP, Thermo Fisher Scientific) for 10 minutes, followed by two 3-minute washes in TBST. Non-specific binding was blocked using 5% Normal Goat Serum (005-000-001, Jackson ImmunoResearch Laboratories, Inc.) for 30 minutes, followed by a 10-minute incubation with Protein Blocking Solution (TA-060-PBQ, Thermo Fisher Scientific).

Primary antibody incubation was conducted overnight at 4° C. with 200× diluted mTT-01 antibody, shielded from light. Post-incubation, sections were washed three times in TBST for 3 minutes each. Amplification of primary antibody binding was performed using Primary Antibody Amplifier Quanto (TL-060-QPB) for 10 minutes, followed by three more TBST washes. Detection was achieved using HRP Polymer Quanto (TL-060-QPH) for 10 minutes, and additional three TBST washes.

Visualization of antigen-antibody complexes was performed using DAB Substrate Kits (TA-060-QHSX and TA-002-QHCX, Thermo Fisher Scientific) for 5 minutes. Slides were then rinsed in tap water for 5 minutes, counterstained with Surgipath Hematoxylin Gill II (3801522, Leica) for 2 minutes, and washed again in tap water for 5 minutes. Dehydration was performed through two 5-minute immersions in 100% alcohol, followed by two 5-minute clearings in Xylene. The slides were then mounted using Surgipath Micromount Mounting Medium (3801731, Leica).

### 1.3 CAR Constructs and Production of CAR T-Cells

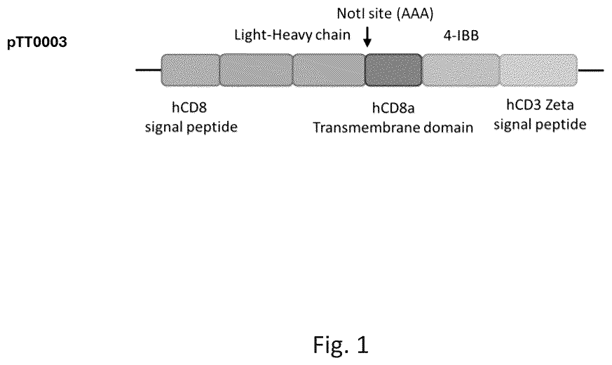

CARs were constructed and expressed based on the humanized anti-PD-L1 monoclonal antibody. Specifically, the plasmid contained humanized anti-PD-L1 scFv sequences (VL to VH), hCD8 hinge, transmembrane domain, 4-1BB co-stimulatory domain, and CD3ζ signaling domain was synthesized by Genescript (NJ, US). And original 3rd generation transfer plasmid, pALD-LentiEGFP was obtained from Aldveron (ND, US). After two plasmids were digested in BstEII/NheI, the CAR sequence was incorporated into BstEII/NheI digested backbone of transfer plasmid using DNA Ligation Kit, Mighty Mix (Takara Bio, Shiga-ken, Japan) and confirm the sequencing using colony PCR and sanger sequencing. The structure of the CAR constructs is shown in FIG. 1. The PD-L1 specific second-generation CARs were designed to harbor a single-chain variable fragment (scFv) linked in the order of VL to VH followed by the hCD8 hinge, transmembrane domain, 4-1BB co-stimulatory domain, and CD3ζ signaling domain. The scFv sequences were obtained from the TT-01 humanized monoclonal antibody against PD-L1. Primary human CD3+ T cells were isolated from the PBMC of healthy donors using CD3 MicroBeads (Miltenyi Biotec, North Rhine-Westphalia, Germany) and stimulated by T cell transAct (Miltenyi Biotec, North Rhine-Westphalia, Germany) with 100 IU/ml of recombinant IL-2 (Miltenyi Biotec, North Rhine-Westphalia, Germany). After 3 days stimulation, the activated CD3+ T cells were transduced with polybrene, Vectofusion-1 (Miltenyi Biotec, North Rhine-Westphalia, Germany) or RetroNectin (Takara Bio, Shiga-ken, Japan) in decided functional MOI (Multiplicity of Infection) of lentivirus bearing CAR gene. Lentivirus containing supernatant was removed at one-or two-days post-infection. The culture medium of CAR T-cells was replaced every 2-3 days with fresh 100 IU/mL recombinant IL-2-containing media. The levels of CAR expression on T cells were measured at 12 days after transduction using flow cytometry. In vitro Cytotoxicity and Safety assay were performed with CAR T-cells at day 12 to 14.

### 1.4 Cell Culture

All cancer cell lines were sub-cultivated every 2 to 3 days using Trypsin-EDTA solution (Gibco, MD, US) and were then incubated in a humidified incubator with 5% CO₂ at 37° C. The base medium for the colorectal cancer cell line COLO 205 and the pancreatic cell line BxPC-3 was RPMI-1640 medium. The pancreatic cell line MIA-CaPa and the brain cell line LN229 were cultured in DMEM medium (Gibco, MD, US). Among them, LN229 was specifically cultured in high-glucose DMEM. Other cell lines, such as the lung cell line SK-LU-1, the brain cell line U87MG, and the liver cell lines HepG2 and PLC, were cultured in MEM medium (Gibco, MD, US). All base media required supplementation with 10% heat-inactivated fetal bovine serum and 1% Penicillin-Streptomycin. Breast cancer cell lines (MDA-MB-231, MDA-MB-231 PD-L1^{KO}, and MCF-7) were maintained in DMEM/F12 (Gibco MD, US) supplemented with 10% heat-inactivated fetal bovine serum and 1% Penicillin-Streptomycin.

### 1.5 In Vitro Safety Assay

The safety of CAR-T cells was demonstrated by co-culturing CAR-T cells with activated Pan T cells, which express PD-L1 on their surface. T cells were enriched from frozen PBMCs using human CD3 microbeads (Miltenyi Biotec, Bergisch Gladbach, Germany) and then activated with T cell TransAct (Miltenyi Biotec, Bergisch Gladbach, Germany.) They were cultured in TexMACS medium (Miltenyi Biotec, Bergisch Gladbach, Germany) containing 100 IU/mL rhIL-2 (Miltenyi Biotec, Bergisch Gladbach, Germany) for 24 hours. To confirm PD-L1 expression before co-culturing, Pan T cells were incubated with Recombinant Anti-PD-L1 antibody 28-8 (ab205921, Abcam, Cambridge, UK) in FACS buffer (2% FBS in PBS) at 4° C. for 30 minutes. The cells were then washed with FACS buffer and incubated with Alexa Fluor® 647 Rabbit Anti-Human IgG secondary antibody (Thermo Fisher, MA, US) in FACS buffer for 30 minutes at 4° C. 7-amino-actinomycin D (7-AAD) (Biolegend, CA, US) staining was used to distinguish viable cells before analysis. Following confirmation, activated Pan T cells were labeled using the CellTrace™ Violet Cell Proliferation Kit (Thermo Fisher, MA, US), following the manufacturer's instructions. Subsequently, 3×10⁵ cells/well were co-cultured with CAR-T cells for different timepoints at different E:T ratios. The supernatants from each group were collected at predetermined time points to detect cytokine release, confirming the safety of the CAR-T cells. In addition to 7-amino-actinomycin D (7-AAD), eBioscience™ Fixable Viability Dye eFluor™ 780 (Thermo Fisher, 65-0865-14) was also used to distinguish viable cells before analysis.

### 1.6 Luciferase Assay

MDA-MB-231^{GFP/Luc} cell line was generated by transducing MDA-MB-231 cells with a lentivirus encoding green fluorescent protein (GFP) and luciferase (Luc). The stable surface expression of GFP on MDA-MB-231^{GFP/Luc} cells was confirmed through flow cytometry. For the assay, MDA-MB-231^{GFP/Luc} cells (1×10⁴ cells/well, in a 96-well white plate) were co-incubated with TT0003 CAR-T, AtezoCAR-T, and CD3+ T cells for required time at the required E:T ratios. The ONE-Glo™ Luciferase Assay System (Promega, WI, US) was utilized to assess the luciferase activity from MDA-MB-231^{GFP/Luc} cells post co-incubation at desired time points. Luminescence (RLU) was recorded using a SpectraMax iD5 Microplate Reader Luminometer (Molecular Devices, CA, US) and normalized to control wells without co-incubation (E:T ration of 0:1).

1.7 In Vitro Cytotoxicity Assay

The cytotoxic potential of CAR-T cells was determined using the Cell Counting Kit-8 (CCK-8) assay (Dojindo, MD, US). Various cell lines including A549, LN229, BxPC-3, U87-MG, SK-LU-1, HepG2, PLC, MCF-7, MDA-MB-231, MDA-MB-231^{GFP/Luc}, and MIDA-MB-231 PD-L1^{KO} (1×10⁴ cells/well, in a 96-well clear plate) were co-cultured with TT0003 CAR-T cells at required E:T ratios for necessary time points. Following co-culture, CAR-T cells were separated from the target cells through dual PBS washes. Tumor cell viability post-treatment was evaluated using CCK-8, with absorbance measured by the SpectraMax iD5 Microplate Reader (Molecular Devices, CA, US). Cytotoxicity was calculated using the formula: % lysis=1−(CCK-8 OD_{450 nm} from wells treated with CAR-T cells)/(CCK-8 OD_{450 nm} from untreated wells)×100%.

### 1.8 Cytokine ELISA Analysis

Collected the conditioned medium after co-culture of tumor cells and CAR-T cells. The IFN-γ, IL-2, and IL-6 cytokines release were evaluated via DuoSet ELISA kit (R & D Systems, MN, US). Sandwich ELISA proceed according to the manufacturer's instructions as follows. Prepare the microplate by coating it with diluted capture antibody overnight at room temperature (RT). Wash and block the plate at RT before adding samples or standards. Followed by detection antibody addition, washing, and Streptavidin-HRP B incubation. Complete the assay with TMB substrate solution addition, stopping the reaction, and measuring optical density using the SpectraMax iD5 Microplate Reader absorbance meter (Molecular Devices, CA, US) set to 450 nm. Perform wavelength correction by 540 nm or 570 nm for accuracy.

### 1.9 Fluorescence-Activated Cell Sorting (FACS) Binding Assay

To evaluate the expression levels of PD-L1 in cancer cell lines, cells were incubated with hTT-01 monoclonal antibody or Recombinant Anti-PD-L1 antibody 28-8 (ab205921, Abcam, Cambidge, UK) in FACS buffer (2% FBS in PBS) at 4° C. for 30 min. The cells were then washed with FACS buffer and incubated with Alexa Fluor® 647 Goat Anti-Human IgG or Alexa Fluor® 647 Goat Anti-Rabbit IgG secondary antibody (Thermo Fisher, MA, US) in FACS buffer for 30 minutes at 4° C. 7-amino-actinomycin D (7-AAD) staining was used to distinguish viable cells before analysis. The transduction efficiency and the subset of CAR T-cells were measured using flow cytometry¹⁰. Briefly, CAR T-cells were collected and blocked with Human TruStain FcX (Biolegend, CA, US) in FACS buffer at 4° C. for 15 min. For detection of scFv expression level, followed by staining with biotinylated PD-L1 (ACROBiosystem, DE, US) at 4° C. for 30 min. After washing, the cells were incubated with R-Phycoerythrin Streptavidin (Jackson ImmunoResearch, PA, US) in FACS buffer at 4° C. for 30 min. For T cell subset analysis, cells were probed with monoclonal antibodies against human CD3, CD4, CD8, CD45RA, CD45RO, and CD62L (Biolegend, CA, US) at 4° C. for 30 min after Fc blocked. The live cells were determined using eBioscience™ Fixable Viability Dye eFluor™ 780 (Thermo Fisher, MA, US). The stained cells were analyzed by an LSRFortessa™ (BD Biosciences, NJ, US), and the data was organized using FlowJo V10 software. To determine the memory function of CAR T-cells, blood, spleen, and Bone marrow were collected from CAR T-cells treated mice after tumor disappear. The staining procedures and antibody panels were followed by the condition in transduction efficiency and the subset of CAR T-cells. In addition to 7-amino-actinomycin D (7-AAD), eBioscience™ Fixable Viability Dye eFluor™ 780 (Thermo Fisher, 65-0865-14) was also used to distinguish viable cells before analysis.

### 1.10 NK and CAR-NK Cell Bioprocess

Enriched NK cells obtained from healthy donor's PBMCs were transduced with a second-generation CAR targeting PD-L1 through proprietary transduction and cell culture techniques of ARCE Therapeutics. In brief, PBMCs isolated from the peripheral blood of healthy donors were static cultured in medium supplemented with 10% Human Platelet Lysate (HPL) in a 6-well plate. The exchange of medium was conducted every 3-4 days and the enriched NK cells were ready for CAR lentiviral vector transduction by ARCE's proprietary transduction and cell culture techniques.

The culture was continued to expand/split until day28 for cell harvest and storage. At indicated time-points, cells were being sampling out for assessing CAR-NK cell productivity and characteristics.

### 1.11 NK/CAR-NK Cytotoxicity Assays

### 1.11.1 2D Potency Assay

Cytolytic activity of CAR-NK cells against tumor cell lines at different effector-to-target (E:T) ratios was measured using luciferase-based reporter assay after co-culture for 24 hrs. CAR-NK cells were incubated with luciferase-engineered tumor target cells at the indicated E/T ratios for 24 hrs. The luciferase activity of the remaining cell lysates after adding the luciferin solution was measured using the luminescence plate reader. The specific lysis was calculated from the data according to the formula: % specific lysis=100×(experimental release-spontaneous release)/(maximum release-spontaneous release)

### 1.11.2 3D Potency Assay

Tumor cells stably expressing the luciferase reporter were cultured in a 96-well U-bottom plate for 3-4 days to form the spheres, and then were incubated with the CAR-NK cells at the indicated E/T ratios for 24 hrs. The luciferase activity of the remaining cell lysates after adding the luciferin solution was measured using the luminescence plate reader. The specific lysis was calculated from the data according to the formula: % specific lysis=100×(experimental release-spontaneous release)/(maximum release-spontaneous release)

### 1.12 Cytokine Analysis

CAR-NK cells were incubated with MDA-MB-231 breast cancer cells for 24 hrs at the E/T ratio of 2. The co-cultured supernatant was harvested and assayed for cytokine production using LEGENDplex™ Multiplex Assays (BioLegend Inc.).

### 1.13 Effect of Macrophages on CAR-NK Cell Cytotoxicity

Cytolytic activity of gPD-L1 CAR-NK cells and Atezo CAR-NK cells against MDA-MB231_mcherry_Luc spheroids pre-incubated with macrophages (E/T=0.5) for 2 days at the indicated E/T ratios of 0.5, 1 and 2 was measured after co-culture for 24 hrs using luciferase-based reporter assay. Before co-culture, CAR-NK cells were labelled with Green dye: Calcein AM (C3009, Invitrogen) and Cultured macrophages were labelled with Blue dye: Hoechst 33342 (2189158, Invitrogen).

### 1.14 Animal Experiment

Advanced severe immunodeficiency (ASID) (NOD.Cg-Prkdc^{scid}I12rg^{tm1Wj1}/YckNarl) mice were maintained and used in the study. Animal experiments were performed according to protocols reviewed and approved by the Institutional Animal Care and Use Committee (IACUC) at the National Laboratory Animal Center (NLAC), Taipei, Taiwan. To establish breast cancer tumor models, ASID mice were subcutaneously inoculated with MDA-MB-231^{GFP/Luc} cells pre-mixed with Matrigel Matrix at a 1:1 ratio. For CAR T-cell therapy, mice received a single dose of 1×10⁷ CAR T-cells when tumor volumes exceeded 50 mm³. In a separate experiment for gPD-L1 CAR-NK cell therapy, mice were inoculated with 2×10⁶ MDA-MB-231^{GFP/Luc} cells and assigned to receive either saline, untransduced NK cells, or gPD-L1 CAR-NK cells (˜95% CAR⁺) when tumor volumes reached 50 mm³. Treatment involved five doses of 1×10⁷ cells delivered peritumorally twice weekly starting 11 days post-tumor inoculation. Bioluminescence imaging was used to monitor tumor growth before and after cell injections on days 10, 17, 24, 31, 38, and 45, with light output quantified as photons/sec/cm²/sr. Tumor dimensions were measured with calipers, and volumes were calculated using the formula V=½(length×width²). These experiments provided critical insights into the therapeutic potential and specificity of CAR-T and CAR-NK cell therapies in breast cancer xenograft models.

### 1.15 Multi-Cycle Kinetic Analysis of Recombinant PD-L1 to hTT-01

This experiment was performed using a Biacore T200 (GE Healthcare). hTT-01 was first diluted with 1×HBS-EP+(BR100669, GE Healthcare) to obtain a final concentration of 3 μg/mL and captured by Flow cell 2 of a Sensor Chip Protein A (29127556, GE Healthcare). The chip contains MabSelect SuRe ligand on the surface, allowing orientation-specific binding of the Fc region of an antibody.

The assay was performed by using the Kinetic/Affinity wizard. The Flow path was 2-1, as the ligand was injected and captured in Flow cell 2 and Flow cell 1 acted as a reference. A series of concentrations of PD-L1 were injected over the reference and the ligand surface consecutively as the association phase with short dissociation phases in between by injection running buffer. Then regeneration solution was injected as the regeneration phase. All the procedures were conducted at 25° C.

### 1.16 Statistical Analysis

Two independent groups were analyzed using Student's t-test, and multiple groups was performed using one-way ANOVA. The data was presented as mean±SD (standard deviation) and p-values≤0.05 were considered statistically significant. All statistical analyses were performed using GraphPad Prism v8.0 (GraphPad Software, San Diego, CA).

### 2. Results

### 2.1 Antibody Tests

### 2.1.1 Immunohistochemistry Assay

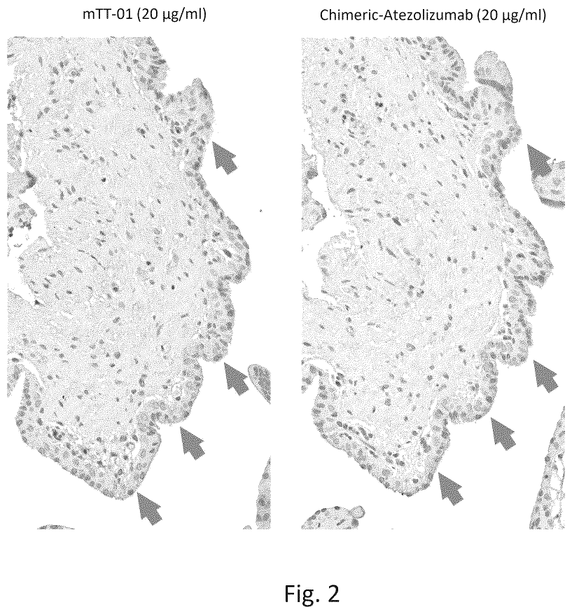

The comparative analysis of binding properties between chimeric-atezolizumab and mTT-01 in human tissues highlights the enhanced specificity of mTT-01, attribute to its interaction with different N-glycosylated sites on PD-L1 compared to those targeted by chimeric-atezolizumab (FIGS. 2-5).

Human placenta, tonsil and breast cancer tissue were used to compare the binding properties of mTT-01 to chimeric-atezolizumab (Anti-hPD-L1-mlgG1 InvivoFit, Cat #: hpdl1-mab9-1, InvivoGen). Chimeric-atezolizumab (Anti-hPD-L1-mIgG1) features the constant region of the mouse IgG1 isotype and the variable region of Atezolizumab. Under the same working concentration (20 μg/ml), positive membrane staining was observed among both mTT-01- and chimeric-atezolizumab-stained placenta tissue (FIG. 2, arrow-pointed). However, the staining intensity was lower in mTT-01-stained tissue (FIG. 2, left panel).

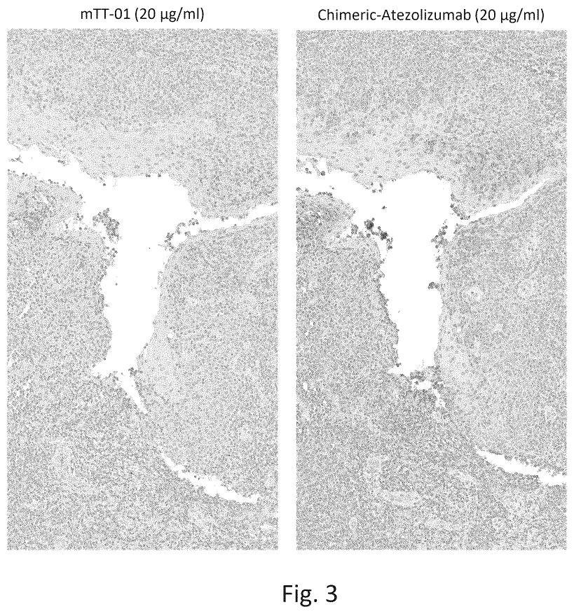

In tonsil tissue, mTT-01 and chimeric-atezolizumab showed similar staining patterns. However, the staining intensity was lower in mTT-01-stained tissue (FIG. 3, left panel).

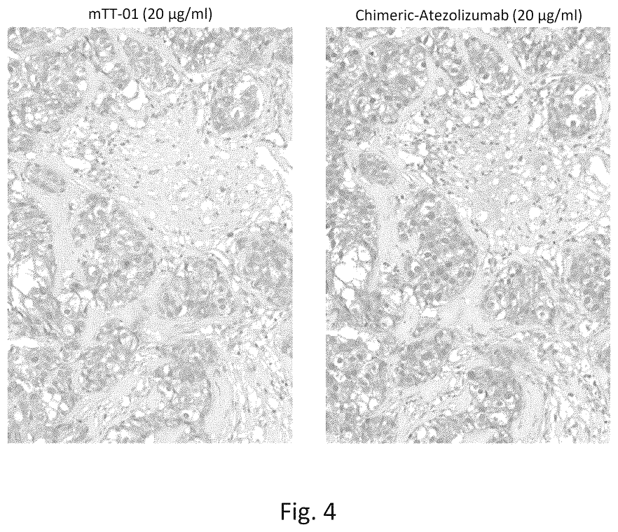

In breast cancer tissue, both mTT-01 and chimeric-atezolizumab showed membrane staining patterns in tumor cells. However, the intensity of cytoplasmic staining was higher in the chimeric-atezolizumab-stained tissue (FIG. 4, right panel). Also, background staining in stroma and nucleus was observed in the chimeric-atezolizumab-stained tissue (Table 7).

TABLE 7
Binding Properties Comparisons Between mTT-01
and Chimeric Atezolizumab in Human Placenta,
Tonsil, and Breast Cancer Tissue.

| | | Chimeric |
|---|---|---|
| (20 μg/ml) | mTT-01 | Atezolizumab |

Breast cancer tissue

| | | | |
|---|---|---|---|
| Tumor cells | Membrane staining | +++ | ++ |
| | Cytoplasmic staining | ++ | +++ |

| | | |
|---|---|---|
| Immune cells | − | + |
| Stroma | − | ++ |
| Nucleus | − | ++ |

Normal tissue

| | | |
|---|---|---|
| Tonsil | + | ++ |
| Placenta | + | ++ |

The binding property comparison between mTT-01 and chimeric-atezolizumab was further investigated using SCMH breast cancer TMA (BRCA-23). A total of 43 breast cancer cores and 44 cancer adjacent normal tissue cores were evaluated. Binding properties of the antibodies are determined by using TPS. The specimen was considered antibody-stained positively if TPS≥50% of the viable tumor cells exhibit membrane staining at any intensity. Binding properties of the antibodies to normal ducts is determined by the same standard that ≥50% of viable ducts show membrane staining at any intensity is defined as positive ductal staining.

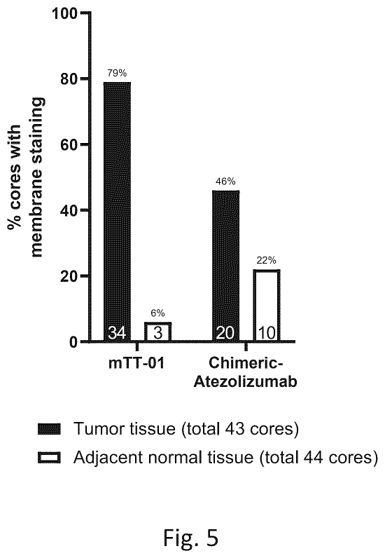

Overall, positive membranous mTT-01 staining was found in 34 out of 43 breast cancer cores (79%, FIG. 5). On the other hand, positive membranous chimeric-atezolizumab staining was found in 20 out of 43 breast cancer cores (46.5%). All (20) breast cancer cores stained positively by chimeric-atezolizumab were also stained positively by mTT-01.

Among 43 breast cancer cores, 26 of them are identified as DCIS core, and the other 17 are invasive carcinoma tissue. Positive membranous mTT-01 staining was found in 22 out of 26 DCIS cores (84.6%), while positive membranous chimeric-atezolizumab staining was found in 17 out of 26 DCIS cores (65.39%). The major difference was found in the positive membranous staining in the invasive carcinoma cores while positive membranous mTT-01 staining was found in 70.6% of cores, only 17.6% of the cores were chimeric-atezolizumab positive.

In cancer adjacent normal tissue, on the contrary, 3 out of 44 cores (3.6%) showed positive membranous mTT-01 staining on normal ducts, while 10 out of 44 cores (22.7%) showed positive membranous chimeric-atezolizumab staining. All (3) adjacent normal cores stained positively by mTT-01 were also stained positively by chimeric-atezolizumab.

### 2.1.2 Binding Affinity Assay

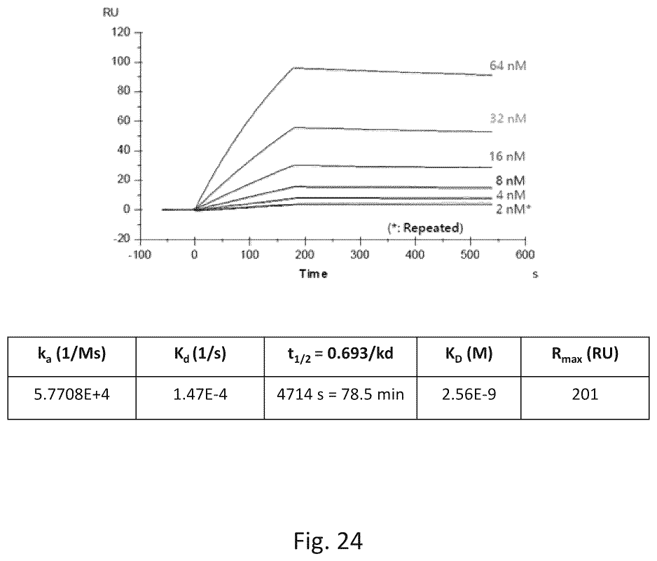

The binding affinity between hTT-01 and PD-L1 was determined using a Biacore T200 (GE Healthcare) with a multi-cycle kinetic analysis. The association rate constant (kₐ) was measured at 5.7708×10⁴ (1/Ms), while the dissociation rate constant (k_{d}) was 1.47×10⁻⁴ (1/s). The calculated half-life (t_{1/2}) of the interaction was 4714 seconds, indicating a stable binding. The equilibrium dissociation constant (K_{D}) was determined to be 2.56 nM, demonstrating a high-affinity interaction between hTT-01 and PD-L1. The maximum response (Rₘₐₓ) was 201 RU, confirming efficient binding to the sensor chip surface. These results indicate that hTT-01 exhibits strong and stable binding to PD-L1, supporting its potential use in therapeutic applications. See FIG. 24.

### 2.2 Establishment of CAR Constructs in CD3+ T Cells

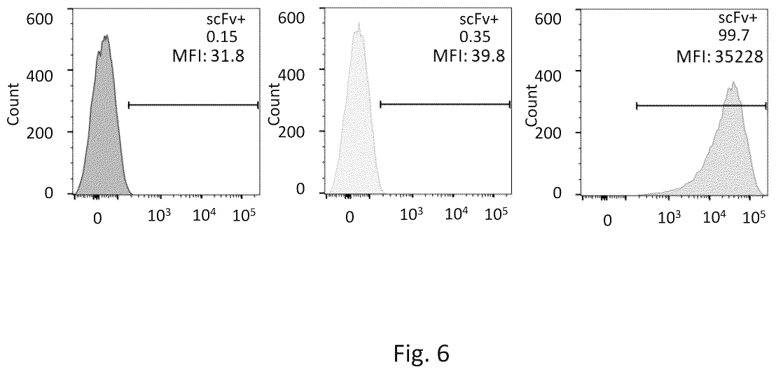

This section of the patent application elaborates on the successful establishment of CAR constructs in primary human CD3+ T cells, as substantiated by FIG. 6. The process involved the transduction of these T cells with a lentiviral vector carrying the TT0003 CAR construct (SEQ ID NO: 25, Table 5).

FIG. 6 shows the establishment of TT0003 CAR constructs in CD3+ T cells. Following the transduction of primary CD3+ T cells with the lentiviral vector, the surface expression of TT0003 CAR was assessed using PD-L1 staining by FACS. The left image served as a reference for normal CD3+ T cells, outlining a cell distribution exceeding the threshold at 0.15%, thereby establishing the baseline CAR expression. In the middle image, the mock group (without transduction) demonstrated a staining of 0.35%. The right image illustrates the embodiment, where populations were divided with the same threshold, revealing over 99.7% of T cells expressing CAR, with an MFI of 35228. This value was significantly higher than the 31.8 MFI observed in normal CD3+ T cells, indicating successful TT0003 expression in CAR-T cells.

### 2.3 Cytotoxicity and PD-L1 Expression Analysis in Cancer Cell Lines with CAR-T Treatment

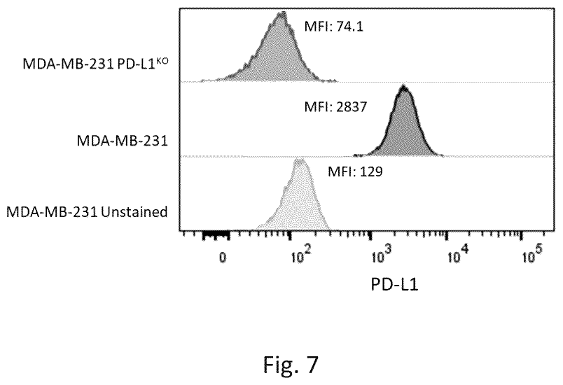

A comprehensive evaluation of CAR-T cells against various cancer cell lines established a crucial correlation between cytotoxicity and PD-L1 expression levels in these cells, as referenced in the figures. Visual comparison between the MDA-MB-231 PD-L1^{KO} cell line and the parental MDA-MB-231 cell line initiated the analysis (FIG. 7). The former lacked PD-L1 expression, while the latter exhibited high PD-L1 expression levels. This comparison laid the groundwork for exploring the impact of PD-L1 on the efficacy of CAR-T cell therapy.

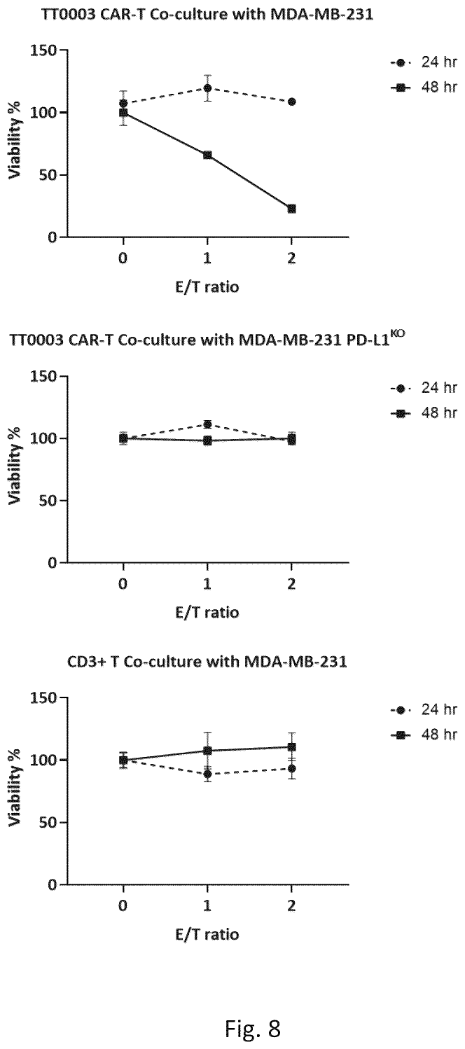

Further investigation into the cytotoxic effects of TT0003 CAR-T cells against MDA-MB-231-derived cell lines revealed intriguing findings (FIG. 8). While the top panel of FIG. 8 demonstrated differential cytotoxicity of TT0003 CAR-T cells against the MDA-MB-231 cell line depending on the E:T ratios and co-culture duration, the middle panel indicated minimal cytotoxicity against the MDA-MB-231 PD-L1^{KO} cell line. These observations underscored the significance of PD-L1 expression in modulating the response to CAR-T cell therapy.

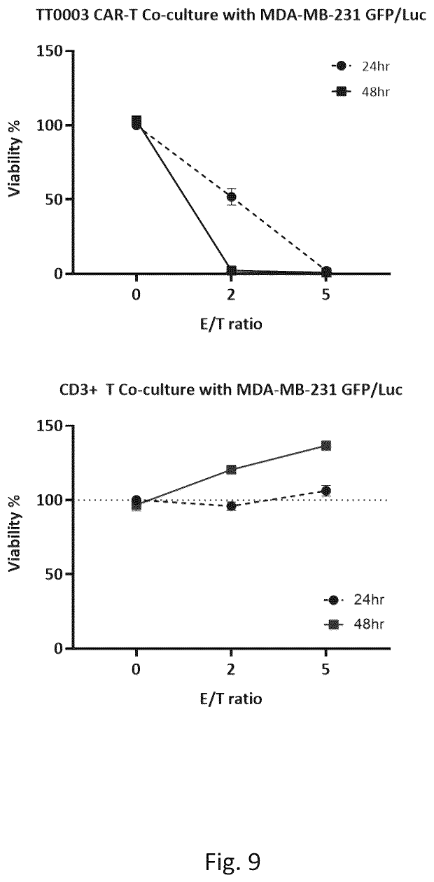

The cytotoxicity of TT0003 CAR-T cells against MDA-MB-231^{GFP/Luc} cells was explored using luciferase assay analysis (FIG. 9). Both E:T ratios of 2:1 and 5:1 demonstrated efficacy in eliminating MDA-MB-231^{GFP/Luc} cells after 24 or 48 hours of co-culture, with no viable cells remaining. These results highlighted the potent cytotoxic effect of TT0003 CAR-T cells against cancer cells expressing PD-L1.

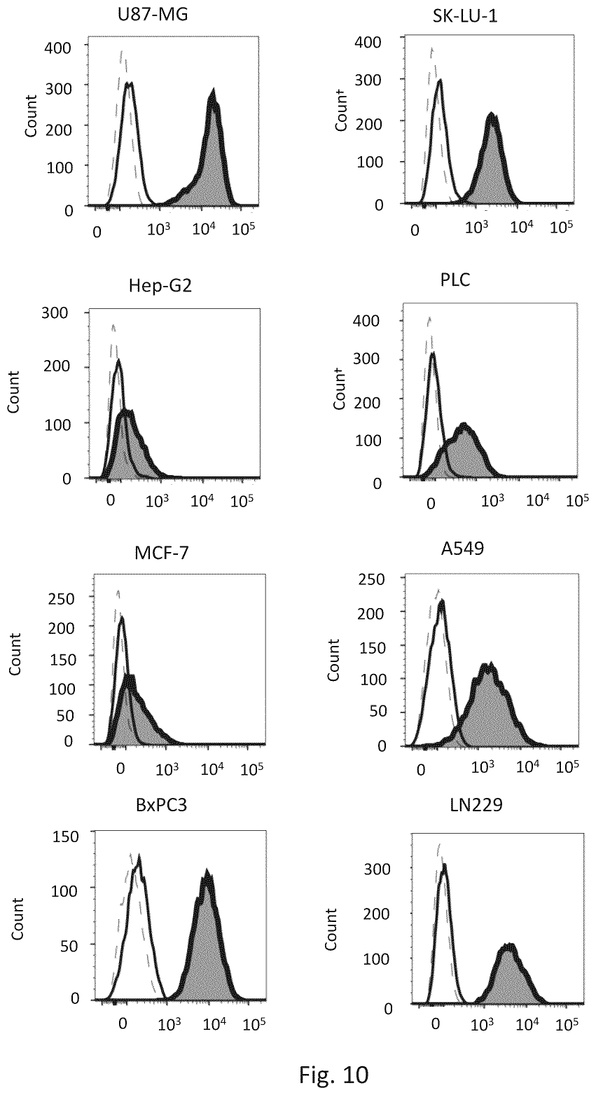

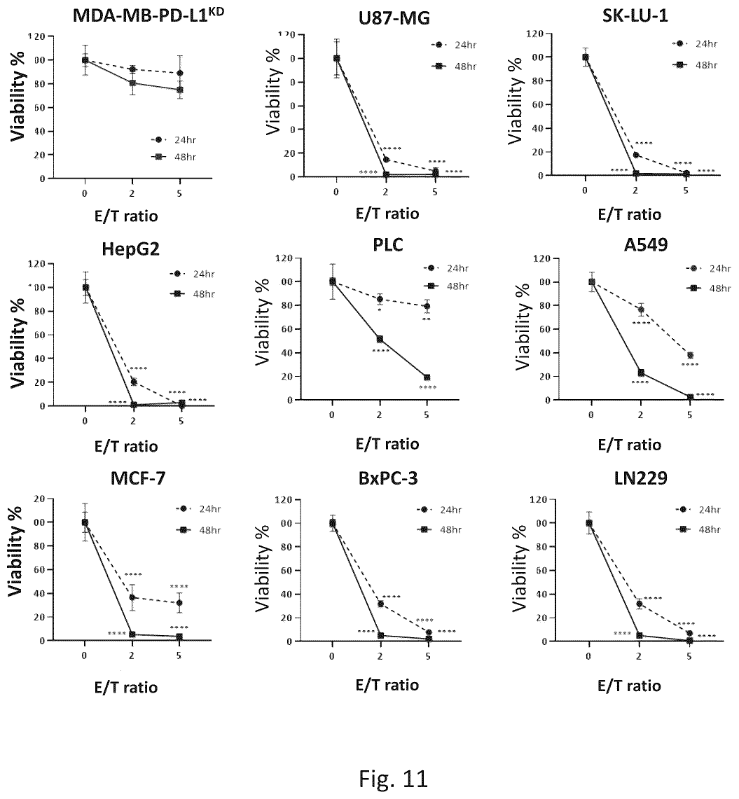

A more in-depth understanding of the variability in response to CAR-T cell therapy was achieved by analyzing PD-L1 expression levels across eight cancer cell lines using FACS detection (FIG. 10). The cell lines were categorized based on their PD-L1 expression levels, providing background information for interpreting the cytotoxicity results. Finally, a comprehensive evaluation of the cytotoxicity of TT0003 CAR-T cells across eight cancer cell lines using the CCK-8 assay revealed consistent patterns (FIG. 11). Strong cytotoxic effects were observed in cell lines with higher PD-L1 expression levels, while lower levels of cytotoxicity were observed in cell lines with lower PD-L1 expression levels. These findings emphasized the potential of TT0003 CAR-T cells in treating breast cancer subtypes with varying PD-L1 expressions, providing valuable insights for future therapeutic strategies.

In summary, the results highlighted the critical role of PD-L1 in modulating the response to CAR-T cell therapy and underscored CAR-T cells as a potential targeted approach for cancer treatment.

### 2.4 Comparative Safety, Stability and Cytotoxicity Analysis of TT0003 CAR-T and AtezoCAR-T Based on Expansion, Viability, and Cytokine Release Profiles

To enhance CAR-T cell therapy for cancer, it's crucial to assess the safety of different CAR-T constructs. This section compares TT0003 and AtezoCAR-T, examining their growth, survival, and cytokine release to evaluate their safety and effectiveness.

### 2.4.1 Expansion Capacity

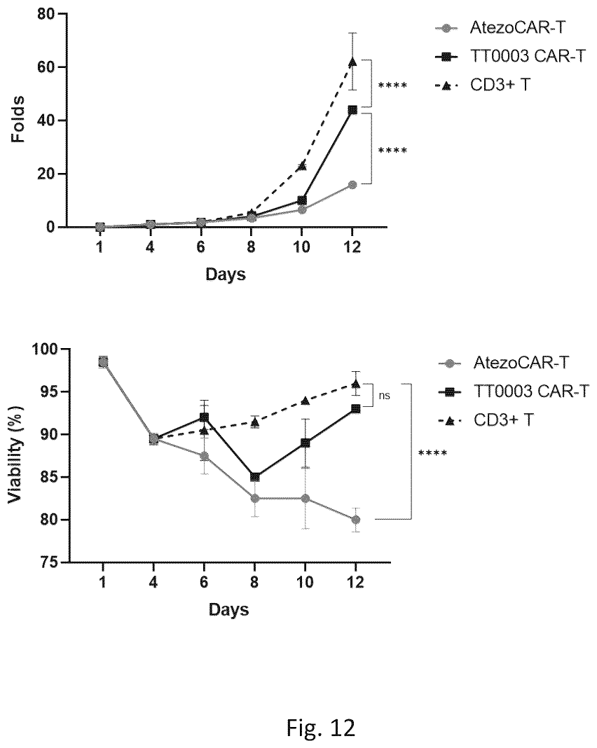

Firstly, FIG. 12 elucidates the expansion capacity of TT0003 and AtezoCAR-T cells over a 12-day culturing period. TT0003 demonstrated a markedly higher expansion fold (˜44 fold) as compared to AtezoCAR-T (˜16 fold), closely aligning with the expansion observed in the control CD3+ T cells (˜62 fold) (FIG. 12, top panel). The viability of these CAR-T cells, as depicted in FIG. 12, bottom panel, further supported their safety profiles. While AtezoCAR-T cells showed a decrease in viability to 80% after 12 days of culturing, TT0003 and control CD3+ T cells demonstrated the ability to maintain over 90% viability. This higher viability indicates a more stable expansion process for TT0003, suggesting a lower propensity for inducing cytotoxicity against non-target cells.

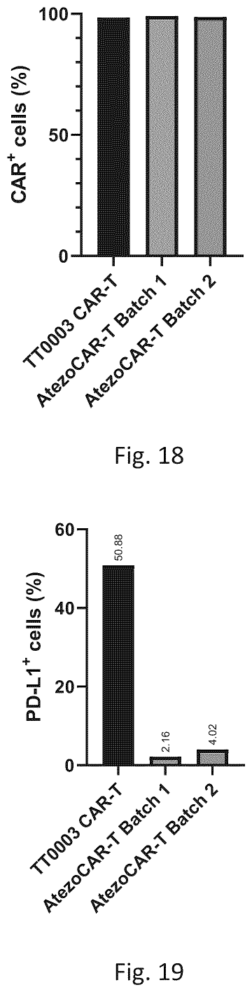

At an MOI of 5, both TT0003 and AtezoCAR-T cells achieved high CAR expression rates exceeding 98%, providing a reliable baseline for comparison (FIG. 18). This consistent expression ensures the relevance of subsequent analyses and underscores the robustness of the manufacturing process.

### 2.4.2 Fratricide Resistance and Specific Cytotoxicity

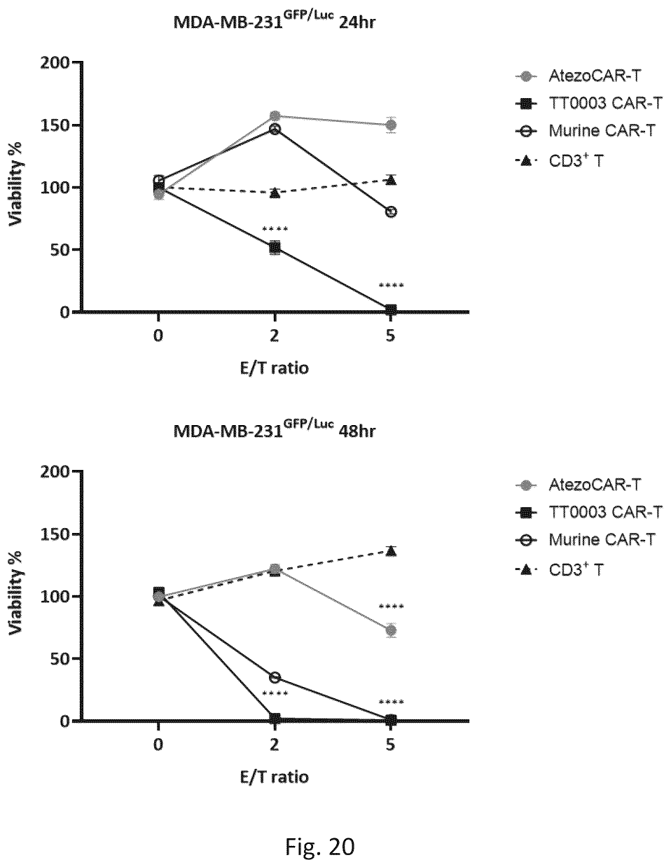

Post-manufacturing analyses revealed stark differences in PD-L1 expression. TT0003 CAR-T cells exhibited higher PD-L1 expression with PD-L1+ cells more than 50% while AtezoCAR-T cells exhibited minimal PD-L1 expression, with PD-L1+ populations remaining below 5% (FIG. 19). This low expression was primarily a result of strong fratricide effects, which caused PD-L1-expressing T cells to die^{7,11}. This fratricide is an inherent characteristic of AtezoCAR-T cells and reflects the low specificity of anti-PD-L1 mechanisms compared to TT0003 CAR-T cells. TT0003 CAR-T cells, in contrast, displayed significantly lower levels of fratricide, maintaining stability and functionality (FIG. 12 and FIG. 20). Cytotoxicity tests against the MDA-MB-231 cancer cell line demonstrated rapid and efficient activity by TT0003 CAR-T cells within 24 hours (FIG. 20, top panel), while AtezoCAR-T cells required up to 48 hours and an E:T ratio of 5 to achieve comparable effects (FIG. 20, bottom panel). These results emphasize the advanced therapeutic performance and robust stability of TT0003 CAR-T cells, showcasing their potential for precise and reliable clinical applications. In contrast, mTT-01 based murine CAR-T cells required 48 hours to reach the level of cytotoxicity observed with TT0003 CAR-T cells at 24 hours (FIG. 20).

### 2.4.3 IFN-γ Release Specificity

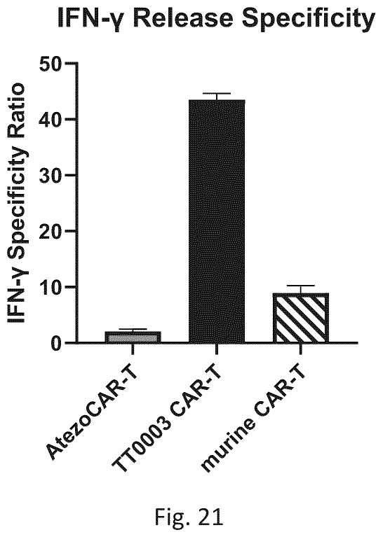

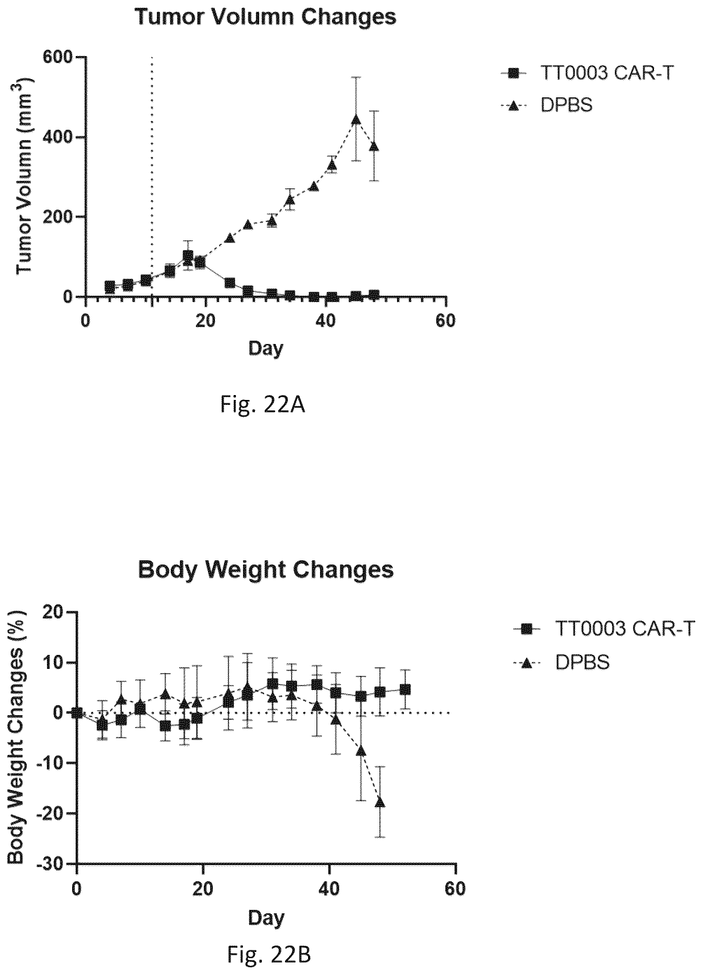

In an in vitro experiment, three types of CAR-T cells-AtezoCAR-T, TT0003 CAR-T, and murine CAR-T-were co-cultured with CD3+ T cells or MDA-MB-231 tumor cells for 48 hours under the same effector-to-target (E/T) ratio of 2. The levels of interferon-gamma (IFN-γ) secreted into the culture medium were measured. To assess the specificity of CAR-T cells toward tumor cells, the IFN-γ concentration from the co-culture of CAR-T cells with MDA-MB-231 tumor cells was divided by the IFN-γ concentration from the co-culture with CD3+ T cells. The IFN-γ Release Specificity Ratio values observed were 2 for AtezoCAR-T, 44 for TT0003 CAR-T, and 9 for murine CAR-T (FIG. 21). Notably, TT0003 CAR-T demonstrated the highest tumor specificity, with a specificity ratio of 44, significantly surpassing both AtezoCAR-T and murine CAR-T. This result highlights the superior specificity of TT0003 CAR-T in distinguishing tumor cells from normal CD3+ T cells, reinforcing its potential for enhanced therapeutic efficacy and reduced on-target, off-tumor effects.

This analysis demonstrates the superior safety, stability, and therapeutic performance of TT0003 CAR-T cells over AtezoCAR-T cells. These results suggest TT0003 CAR-T cells as a safer and more effective option for clinical CAR-T therapies.

### 2.5 Production and Characterization of gPD-L1 CAR-NK Cells

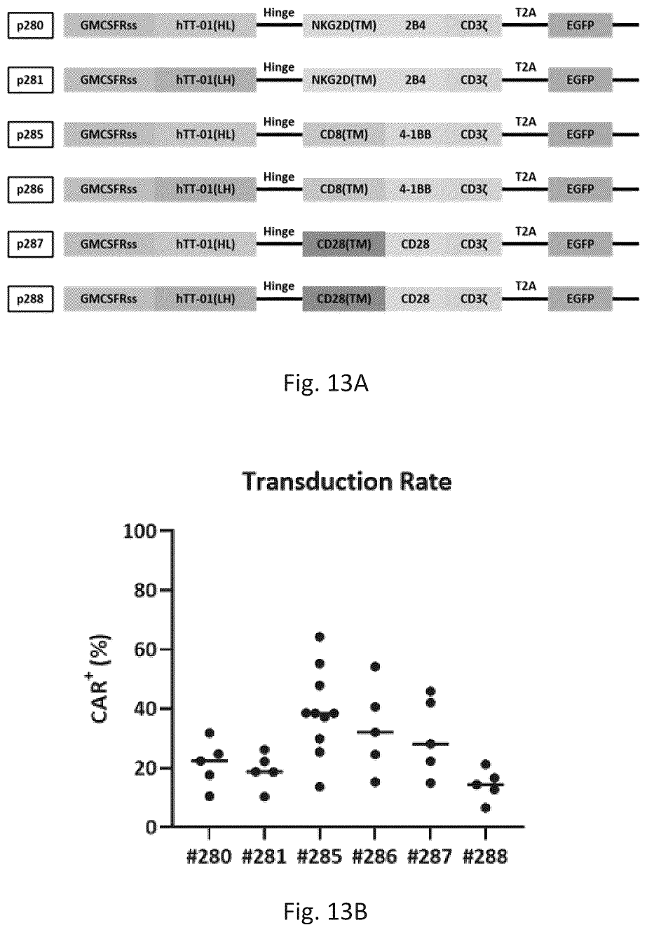

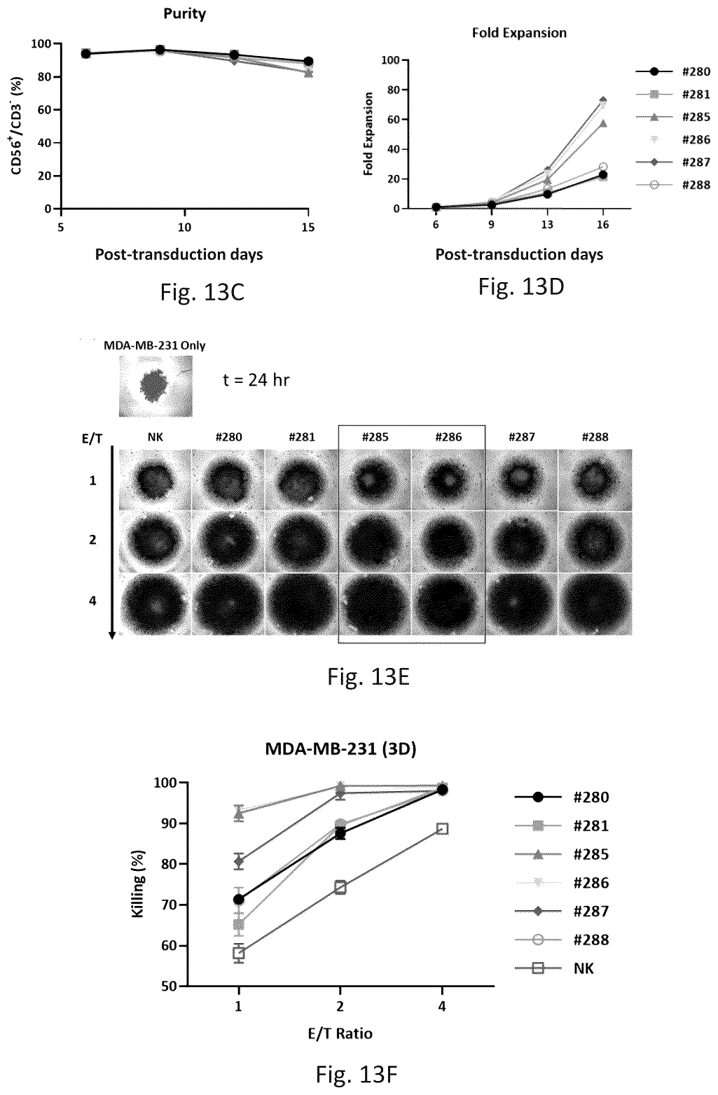

gPD-L1 CAR gene was designed in 2^{nd} generation CAR format with optimization of the hTT-01 based single-chain variable fragment (scFv) V_{H}-V_{L} orientation combined with the co-stimulatory domain of CD28, 4-1BB or 2B4. Each CAR molecule consists the extracellular antigen-interacting domains of scFv in V_{H}-V_{L} or V_{L}-V_{H} orientation, hinge from CD8 or CD28, transmembrane domains derived for CD8, CD28 or NKG2D, and one intracellular costimulatory (ICS) domain derived from CD28, 4-1BB or 2B4, followed by a C-terminal CD3ζ activation domain (FIG. 13A). All six gPD-L1 CAR genes packaged in lentiviral vectors were successfully delivered into primary NK cells by proprietary CAR-NK cell bioprocess, and these CAR-NK cell clones were assessed for transduction efficiency, viability, cell expansion and target cell cytotoxicity. All six CAR-NK cell clones were successfully produced, and clone p285, p286 and p287 were shown to have better transduction efficiencies and cell expansion folds than others (FIGS. 13B, 13C and 13D). In addition, clone p285 and p286 exhibited the superior cytotoxicity against MDA-MB-231 cells (PD-L1 high) by both the image-based cytotoxicity analysis (FIG. 13E) and the luciferase-based cytotoxicity assay (FIG. 13F). The results supported that the designed CAR gene of the gPD-L1 (HL or LH)-CD8-41BB-CD3z format, specifically, represents a favorable configuration within the context of peripheral blood (PB)-derived NK cells.

### 2.6 gPD-L1 CAR-NK Cells are superior to Atezo CAR-NK Cells in Productivity and Target Cell Cytotoxicity

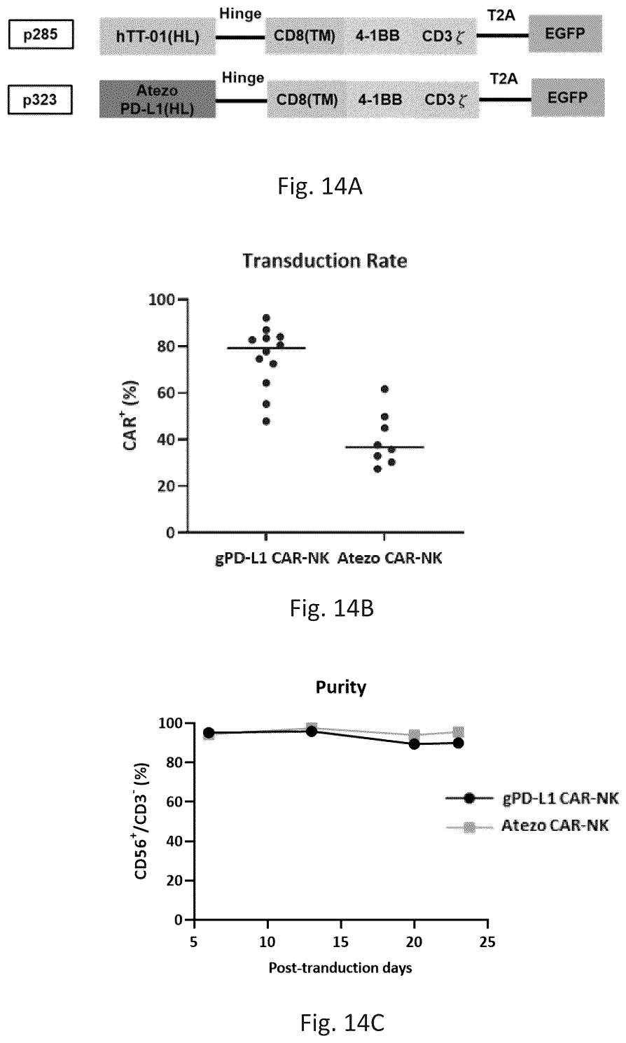

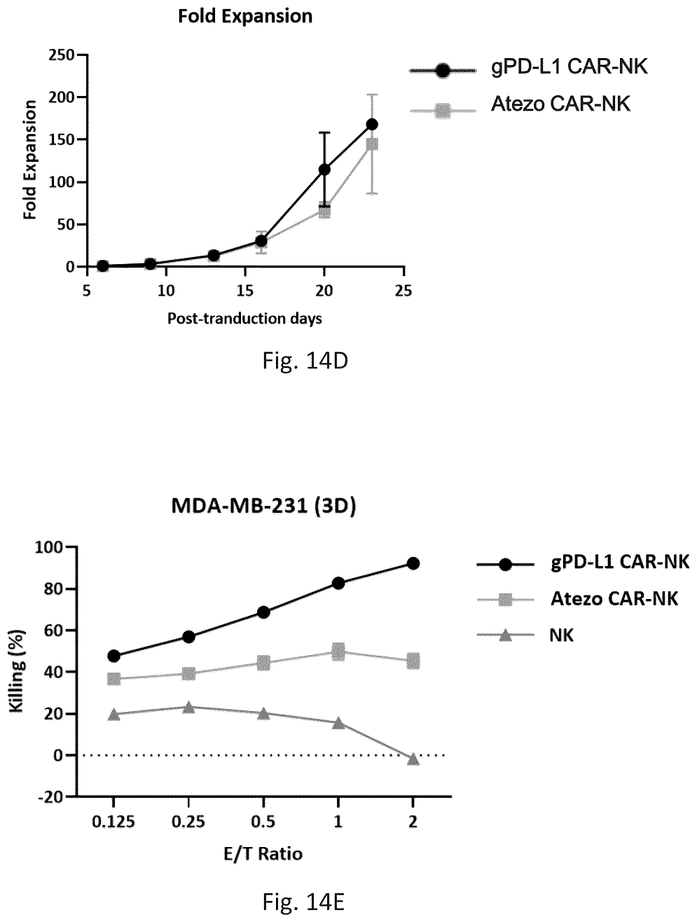

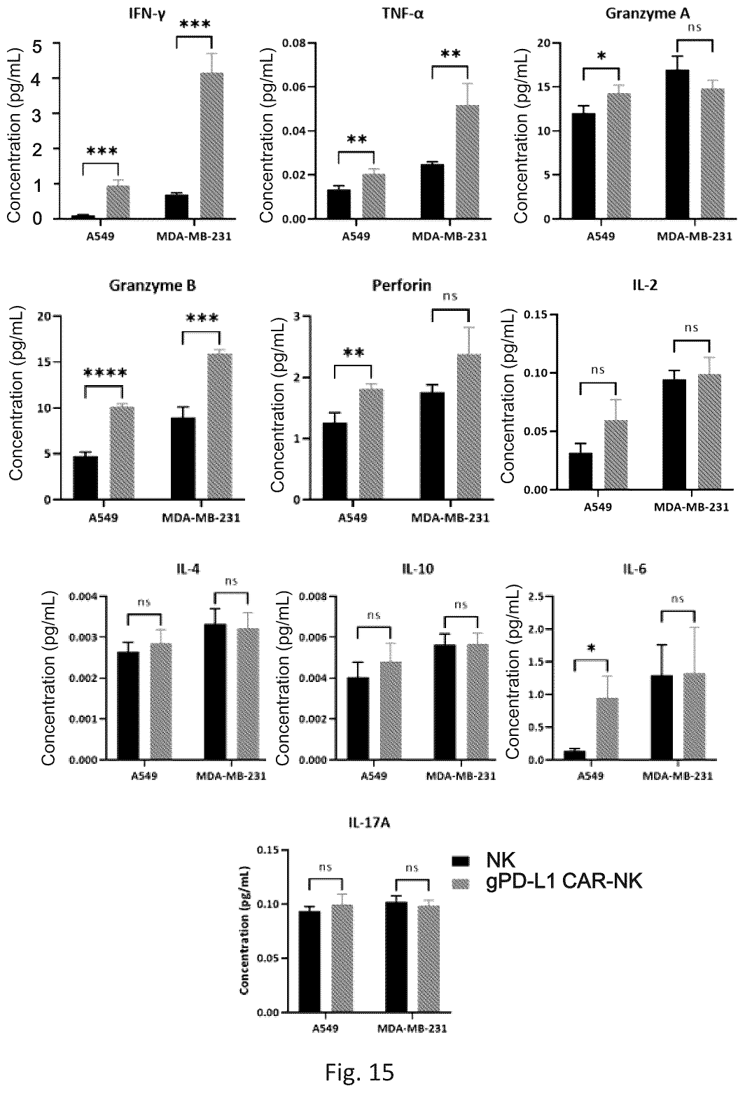

As the reference for gPD-L1 CAR (p285), the anti-PD-L1 antibody (Atezolizumab)-based scFv CAR gene (p323) was designed (FIG. 14A) and proceeded for lentiviral vector production. Both gPD-L1 CAR-NK cells and Atezo CAR-NK cells were produced by proprietary CAR-NK bioprocess and then assessed by transduction efficiency, cell expansion and target cell cytotoxicity. By comparison, gPD-L1 CAR-NK cells were demonstrated to have higher transduction rate of ˜80% and expansion folds (FIGS. 14B, 14C and 14D), and better cytotoxicity of 30% difference at E/T=1 than Atezo CAR-NK cells (FIG. 14E). Analysis of CAR-NK cell phenotypes had revealed that both gPD-L1 CAR-NK cells and Atezo CAR-NK cells expressed substantial levels of NK cell marker, CD56 throughout the CAR-NK cell bioprocess and shared similar expression patterns of activation and inhibition markers, such as CD16, DNAM-1, NKG2D, NKp30, NKp44 and NKP46 (data not shown). In the aspect of cytokine release profiles, gPD-L1 CAR-NK cells co-cultured with MDA-MB-231 or A549 at an E/T ratio of 2 demonstrated the relative high production of INF-γ, TNF-α, Granzyme A, Granzyme B and perforin as compared to their paired NK cells (FIG. 15).

### 2.7 gPD-L1 CAR-NK Cells are Superior to Atezo CAR-NK Cells in Lysing Certain PD-L1^{high} Tumor Cell Lines and Being Resistant to Macrophage Interference

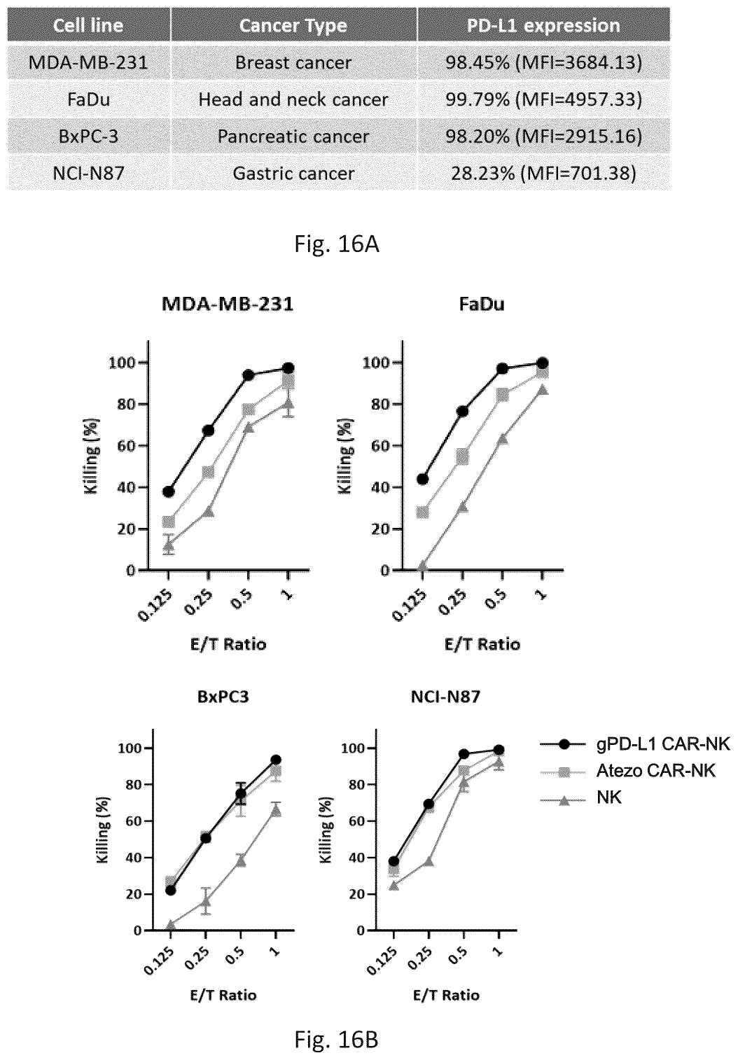

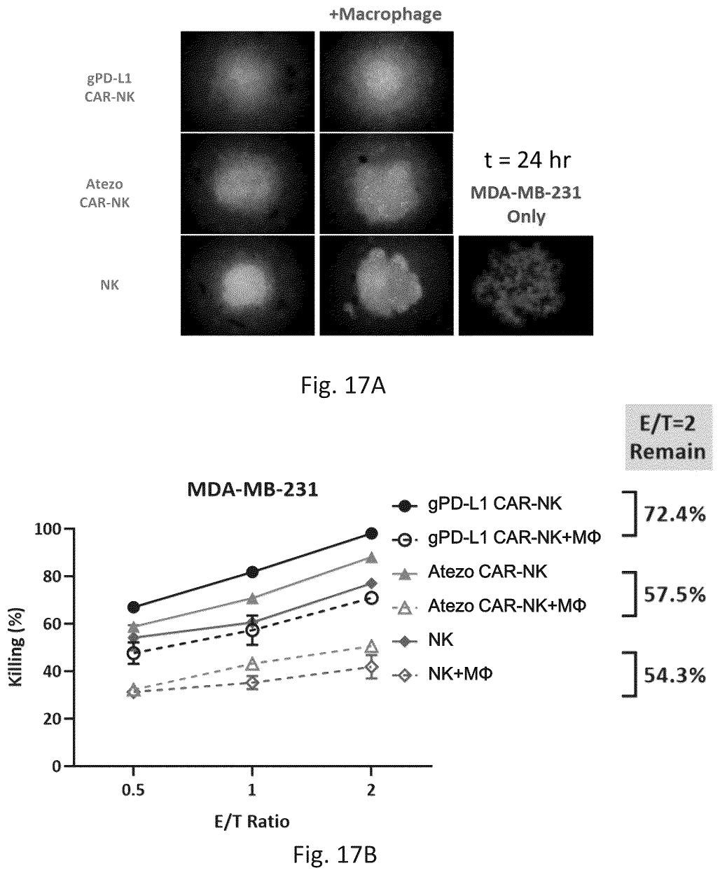

In addition to MDA-MB231 (breast cancer cell), the cytotoxicity assessment of gPD-L1 CAR-NK cells and Atezo CAR-NK cells was further conducted with Fadu (head and neck cancer), BxPC3 (pancreatic cancer cell) and NCI-N87 (gastric cancer cell). On average, MDA-MB-231, FaDu, and BxPC3 exhibited high levels of PD-L1 expression (99%) while NCI-N87 showed low levels of PD-L1 expression (28%) (FIG. 16A) gPD-L1 CAR-NK cells were shown to exhibit better cytotoxicity of over 20% at E/T ratios ranging from 0.125 to 0.5 than Atezo CAR-NK cells against MDA-MB-231 and FaDu, but not BxPC3 cells or NCI-N87 cells though low levels of PD-L1 antigens were expressed on NCI-N87 cells (FIG. 16B). In the context of macrophage-associated tumor microenvironment (TME), a significant reduction of macrophage numbers was found in the treatment with Atezo CAR-NK cells as compared to the treatment with gPD-L1 CAR-NK cells (FIG. 17A). In addition, gPD-L1 CAR-NK cells were less interfered by macrophages to mediated target cell cytotoxicity, probably due to the preferential binding of glycosylated PD-L1 antigens on MDA-MB231 cells (FIG. 17B). Incubation of target tumor cells with macrophages (allogenic) resulted in a 50% reduction in the cytotoxicity of Atezo CAR-NK cells, but had only a 30% reduction in the cytotoxicity of gPD-L1 CAR-NK cells.

### 2.8 In Vivo Efficacy of TT0003 CAR-T in Xenograft Mouse Model

The in vivo impact of TT0003 CAR-T cells on tumor growth was studied using a xenograft mouse model. TT0003 CAR-T cells demonstrated a strong ability to suppress tumor growth, with significant reductions in tumor size observed after administration. Tumors, initially injected and grown to 50 mm³, began to decrease in size around 6 days post-treatment and became undetectable by approximately 30 days post-treatment (FIG. 22A). Treated mice maintained body weight fluctuations within 10% throughout the 52-day study period, while the DPBS control group exhibited rapid weight loss starting around 40 days after tumor injection (FIG. 22B). These results highlight the potential therapeutic benefit of TT0003 CAR-T cells, demonstrating dose-dependent efficacy and providing critical data for further development and optimization as a targeted therapeutic approach in oncology. The findings support TT0003 CAR-T therapy as a promising candidate for advancing effective cancer immunotherapies.

### 2.9 gPD-L1 CAR-NK Cells Inhibit Tumor Growth of MDA-MB-231 In Vivo

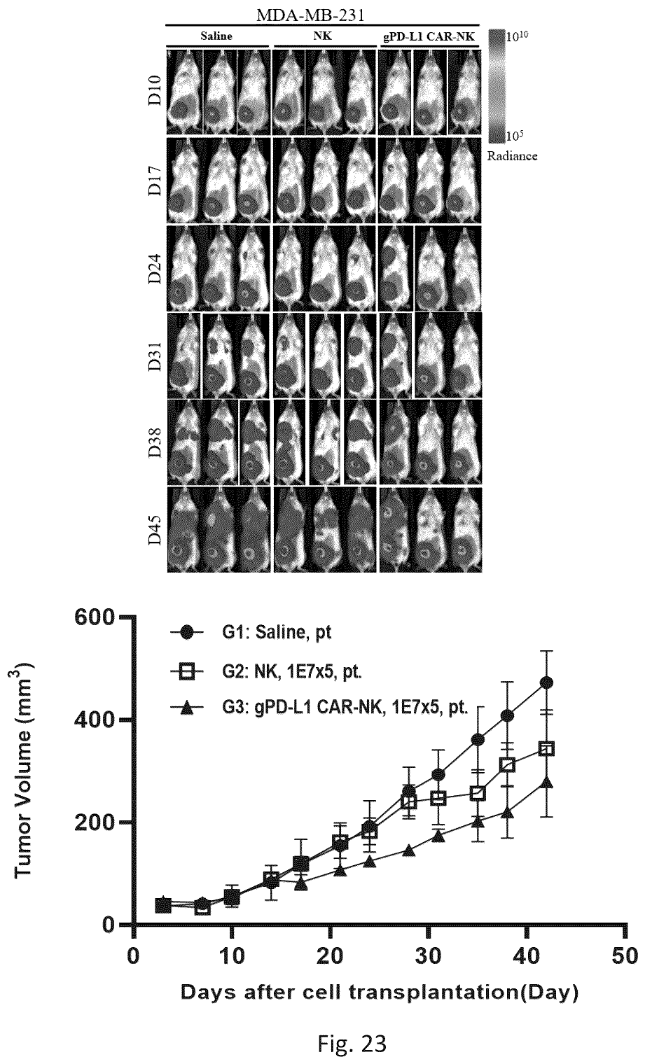

An orthotopic xenograft model of Triple-negative breast cancers (TNBC) cell line MDA-MB-231 was generated by implanting 2×10⁶ luciferase-labeled PD-L1 expressing MDA-MB-231 cells (FIG. 7) into the mammary fat pad in advanced severe immunodeficiency (ASID) mice to study tumor growth (FIG. 23). Untransduced NK cells and gPD-L1 CAR-NK cells were prepared as described in 2.5. Mice inoculated with the tumor cells were treated via peritumoral injection of three different materials: saline, untransduced NK cells or gPD-L1 CAR-NK cells. BLI and tumor volume measurement demonstrated that the tumor burdens in mice receiving gPD-L1 CAR-NK cells were obviously reduced comparing to the control groups. Quantitative analysis of tumor volumes at the latest evaluated time point (Day 42) demonstrated a statistically significant difference between the group of mice treated with gPD-L1 CAR-NK cells and the control group with saline (P≤0.05). Consequently, the treatment with gPD-L1 CAR-NK demonstrated the antitumor effects in vivo.

### 3. Conclusion

This study presents comprehensive data and analyses underscoring the efficacy and specificity of Chimeric Antigen Receptor (CAR) cells in cancer treatment, including CAR-T cells and CAR-NK cells. The findings offer conclusive evidence supporting the innovative approach and effectiveness of CAR cell therapy, particularly in targeting cancers expressing PD-L1.

Efficacy in Target Recognition and Cytotoxicity: The immunohistochemistry assays conclusively demonstrated the specific binding and targeted action of CAR cells against PD-L1 expressing tumor cells. These results indicate a heightened sensitivity and specificity of CAR cells, making them a potent option for treating PD-L1 expressing cancers.

In Vitro Validation: The successful expression of CAR constructs in primary human CD3+ T cells and the subsequent demonstration of their cytotoxicity in various cancer cell lines validate the in vitro efficacy of CAR cells.

Safety and Specificity: The data presented in this patent application also emphasize the importance of specificity in CAR cell therapy. The differentiation in binding properties of hTT-01 and chimeric-atezolizumab to PD-L1 and the observed selective cytotoxicity of CAR-T cells in PD-L1 expressing cells highlight the potential for minimizing off-tumor effects and enhancing the safety profile of this therapy. CAR-NK cells also are demonstrated herein to exhibit superior cell expansion fold, profound target cell cytotoxic activity and resistance to macrophage interference.

In Vivo Validation: In vivo killing of tumor cells by TT0003 CAR-T and gPD-L1 CAR-NK against MDA-MB-231 orthotopic xenografts was demonstrated in ASID mice. Tumor volumes in mice treated with TT0003 CAR-T cells decreased significantly, reaching nearly undetectable levels at the 30 days post-treatment. Tumor volumes in the group treated with gPD-L1 CAR-NK cells remained smaller than those in the mice of the control group at the latest evaluated time point (P≤0.05).

In conclusion, the comprehensive data provided herein affirm the potential of CAR cell therapy as a highly effective, specific, and sustainable treatment modality in the realm of cancer immunotherapy. The specificity in targeting PD-L1 expressing cells, combined with the proven efficacy and safety profile, positions CAR cell therapy as a promising candidate for future clinical applications in oncology, offering new hope for patients battling cancers that express PD-L1.

### REFERENCES

1. PardollDM. The blockade of immune checkpoints in cancer immunotherapy. Nat Rev Cancer. 2012;12(4):252-264. doi:10.1038/nrc3239
2. TwomeyJD, ZhangB. Cancer Immunotherapy Update: FDA-Approved Checkpoint Inhibitors and Companion Diagnostics. AAPS J. 2021;23(2):39. doi:10.1208/s12248-021-00574-0
3. LiC-W, LimS-O, ChungEM, et al. Eradication of Triple-Negative Breast Cancer Cells by Targeting Glycosylated PD-L1. Cancer Cell. 2018;33(2):187-201.e10. doi:https://doi.org/10.1016/j.ccell.2018.01.009
4. BenickyJ, SandaM, Brnakova KennedyZ, et al. PD-L1 Glycosylation and Its Impact on Binding to Clinical Antibodies. J Proteome Res. 2021;20(1):485-497. doi:10.1021/acs.jproteome.0c00521
5. LiC-W, LimS-O, XiaW, et al. Glycosylation and stabilization of programmed death ligand-1 suppresses T-cell activity. Nat Commun. 2016;7(1):12632. doi:10.1038/ncomms12632
6. NeelapuSS, TummalaS, KebriaeiP, et al. Chimeric antigen receptor T-cell therapy—assessment and management of toxicities. Nat Rev Clin Oncol. 2018;15(1):47-62. doi:10.1038/nrclinonc.2017.148
7. BajorM, Graczyk-JarzynkaA, MarhelavaK, et al. PD-L1 CAR effector cells induce self-amplifying cytotoxic effects against target cells. J Immunother Cancer. 2022;10(1):e002500. doi:10.1136/jitc-2021-002500
8. Shimabukuro-VornhagenA, BöllB, SchellongowskiP, et al. Critical care management of chimeric antigen receptor T-cell therapy recipients. CA Cancer J Clin. 2022;72(1):78-93. doi:https://doi.org/10.3322/caac.21702
9. LiuH, MaY, YangC, et al. Severe delayed pulmonary toxicity following PD-L1-specific CAR-T cell therapy for non-small cell lung cancer. Clin Transl Immunol. 2020;9 (10):e1154. doi:https://doi.org/10.1002/cti2.1154
10. HuangH-C, LaiY-J, LiaoC-C, et al. Targeting conserved N-glycosylation blocks SARS-COV-2 variant infection in vitro. eBioMedicine. 2021;74. doi:10.1016/j.ebiom.2021.103712
11. LiuM, WangX, LiW, et al. Targeting PD-L1 in non-small cell lung cancer using CAR T cells, Oncogenesis. 2020;9:72. doi: 10.1038/s41389-020-00257-z

1. A chimeric antigen receptor (CAR) comprising a PD-LI binding domain which comprises
(a) a heavy chain variable region (V_{H}) which comprises a heavy chain complementary determining region (HC CDR1) of SEQ ID NO: 1, a heavy chain complementary determining region 2 (HC CDR2) of SEQ ID NO: 2, and a heavy chain complementary determining region 3 (HC CDR3) of SEQ ID NO: 3; and
(b) a light chain variable region (V_{L}) region which comprises a light chain complementary determining region 1 (LC CDR1) of SEQ ID NO: 4, a light chain complementary determining region (LC CDR2) of SEQ ID NO: 11, and a light chain complementary determining region 3 (LC CDR3) of SEQ ID NO: 6.

2. The CAR of claim 1, wherein the CAR further comprises a hinge domain, a transmembrane domain and an intracellular signaling domain.

3. The CAR of claim 1, wherein the CAR comprises the PD-LI binding domain comprising, from N-terminus to C-terminus, the V_{H} and the V_{L} or the V_{L} and the V_{H}.

4. The CAR of claim 3, wherein the V_{H} and the V_{L} are linked by a linker.

5. The CAR of claim 2, wherein the intracellular signaling domain comprises at least one be selected from CD137 (4-1BB) signal domain, CD28 signal domain, CD27 signal domain, ICOS signal domain, CD3ζ signal domain, 2B4 signal domain and any combination thereof.

6. The CAR of claim 2, wherein the CAR comprises the amino acid sequence of SEQ ID NO: 12 and 13; the CAR comprises the amino acid sequence of SEQ ID NO: 19 or 20; or the CAR comprises the amino acid sequence of SEQ ID NO: 26 or 25; or the CAR comprises the amino acid sequence of P280, P281, P285, P286, P287 and P288 as set forth in Table 6.

7. A nucleic acid molecule comprising a nucleotide sequence encoding a CAR of claim 1.

8. The nucleic acid molecule of claim 7, which is a vector.

9. A cell comprising a nucleic acid molecule of claim 7.

10. The cell of claim 9, wherein the cell is a T cell.

11. The cell of claim 9, wherein the cell is a NK cell.

12. A pharmaceutical composition comprising a cell of claim 9.

13. A method for treating a subject having a disease, disorder or condition associated with an elevated expression of a tumor antigen, comprising
administering to the subject an effective amount of a cell genetically modified to express a CAR of claim 1.

14. The method of claim 13, wherein the tumor antigen is PD-L1.

15. The method of claim 13, wherein the subject suffers from cancer.

16. The method of claim 15, wherein the cancer is selected from the group consisting of breast cancer, colon cancer, head and neck cancer, thyroid cancer, lung cancer, ovarian cancer, pancreatic cancer, gastric cancer, kidney cancer, glioblastoma and leukemia.

17-20. (canceled)

21. An isolated antibody against PD-L1 wherein the antibody comprises (i) a heavy chain variable region (V_{H}) which comprises a heavy chain complementary determining region (HC CDR1) of SEQ ID NO: 1, a heavy chain complementary determining region 2 (HC CDR2) of SEQ ID NO: 2, and a heavy chain complementary determining region 3 (HC CDR3) of SEQ ID NO: 3; and (ii) a light chain variable region (V_{L}) which comprises a light chain complementary determining region 1 (LC CDR1) of SEQ ID NO: 4, a light chain complementary determining region (LC CDR2) of SEQ ID NO: 11, and a light chain complementary determining region 3 (LC CDR3) of SEQ ID NO: 6.

22. The antibody of claim 21, wherein the antibody comprises a V_{H} that comprises SEQ ID NO: 12 and a V_{L} comprising SEQ ID NO: 13.

23. The antibody of claim 21, wherein the antibody is an antigen-binding fragment thereof.

24. The antibody of claim 21, wherein the antibody is humanized.

25. A recombinant nucleic acid comprising a nucleotide sequence encoding an antibody as defined in claim 21.

26. A host cell comprising the recombinant nucleic acid of claim 25.

27. A pharmaceutical composition comprising an antibody as defined in claim 21.

28. A method for treating a subject having a disease, disorder or condition associated with an elevated expression of a tumor antigen, comprising
administering to the subject an effective amount of an antibody as defined in claim 21.

29. The method of claim 28, wherein the tumor antigen is PD-L1.

30. The method of claim 28, wherein the subject suffers from cancer.

31. The method of claim 30, wherein the cancer is selected from the group consisting of breast cancer, colon cancer, head and neck cancer, thyroid cancer, lung cancer, ovarian cancer, pancreatic cancer, gastric cancer, kidney cancer, glioblastoma and leukemia.

32-35. (canceled)


SEQUENCE LISTING (34 sequences)

SEQ ID NO: 1 - protein, 5 aa, synthetic construct
NYVMS

SEQ ID NO: 2 - protein, 17 aa, synthetic construct
TISSGGRYIYYTDSVKG

SEQ ID NO: 3 - protein, 10 aa, synthetic construct
DGSTLYYFDY

SEQ ID NO: 4 - protein, 10 aa, synthetic construct
SASSSVDYMY

SEQ ID NO: 5 - protein, 7 aa, synthetic construct
DTSNLAS

SEQ ID NO: 6 - protein, 10 aa, synthetic construct
QQWSSSPPIT

SEQ ID NO: 7 - protein, 119 aa, synthetic construct
EVMLVESGGALVKPGGSLKLSCAASGFSLSNYVMSWVRQTPEKRLEWVATISSGGRYIYYTDSVKGRFTISRDNARNTLYLQMSSLRSEDTAMYYCARDGSTLYYFDYWGQGTTLTVSS

SEQ ID NO: 8 - protein, 107 aa, synthetic construct
QTVLTQSPAIMSASPGEKVTMTCSASSSVDYMYWYQQKPGSSPRLLIYDTSNLASGVPVRFSGSGSGTSYSLTISRMEAEDAATYYCQQWSSSPPITFGTGTKVELK

SEQ ID NO: 9 - DNA, 357 bp, synthetic construct
  source 1..357: mol_type = other DNA
gaagtgatgctggtggagtctgggggagccttagtgaagcctggagggtccctgaaactctcctgtgcagcttctggattcagtttgagtaactatgtcatgtcttgggttcgccagactccagagaagaggctggagtgggtcgcaaccattagtagtggtggtaggtatatctactatacagacagtgtgaagggtcgattcaccatctccagggacaatgccaggaacaccctgtacctgcaaatgagcagtctgaggtctgaggacacggccatgtattattgtgcaagagacggtagtaccttgtactactttgactattggggccaaggcaccactctcacagtctcctca

SEQ ID NO: 10 - DNA, 321 bp, synthetic construct
  source 1..321: mol_type = other DNA
caaactgttctcacccagtctccagcaatcatgtctgcatctccaggggagaaggtcaccatgacctgcagtgccagctcaagtgtagattacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttccccacccatcacgttcggtactgggaccaaggtggagctgaaa

SEQ ID NO: 11 - protein, 7 aa, synthetic construct
DTSNLAT

SEQ ID NO: 12 - protein, 119 aa, synthetic construct
EVQLVESGGGLVKPGGSLRLSCAASGFSLSNYVMSWVRQAPGKGLEWVATISSGGRYIYYTDSVKGRFTISRDNAKNSLYLQMNSLRAEDTAVYYCARDGSTLYYFDYWGQGTTVTVSS

SEQ ID NO: 13 - protein, 107 aa, synthetic construct
QTVLTQSPATLSLSPGERATLSCSASSSVDYMYWYQQKPGQAPRLLIYDTSNLATGIPARFSGSGSGTDFTLTISSLEPEDFATYYCQQWSSSPPITFGQGTKVEIK

SEQ ID NO: 14 - DNA, 357 bp, synthetic construct
  source 1..357: mol_type = other DNA
gaagtgcagctcgtggagtccggtggcggactcgtgaaacctgggggaagcctgcggttgtcatgtgccgcttcggggttcagcctgtccaactacgtgatgtcgtgggtcagacaggcgccgggaaagggacttgaatgggtggccactattagctcgggcggcagatacatctactacaccgactccgtcaagggccggtttaccatctcccgcgacaacgccaagaactccctgtatctgcaaatgaactcactgcgggcagaggataccgccgtgtactactgcgcccgcgatggttccaccctgtactacttcgactactggggacagggcaccactgtgacggtgtcctca

SEQ ID NO: 15 - DNA, 321 bp, synthetic construct
  source 1..321: mol_type = other DNA
cagaccgtgctgactcagagcccggccacactttccctgtccccgggagaaagggctaccttgtcctgctccgcgtcgtcctccgtggactatatgtactggtaccagcagaagccaggtcaagcccctcgcctgctcatctacgacacttccaacctcgccaccggaatccctgcccggttcagcggatcaggctccggcaccgactttaccctgaccatttcctcgctggagcccgaggatttcgcaacctactactgtcagcaatggtcaagctcgcccccgatcacgttcggccaggggactaaggtcgaaatcaaa

SEQ ID NO: 16 - protein, 21 aa, synthetic construct
MALPVTALLLPLALLLHAARP

SEQ ID NO: 17 - protein, 21 aa, synthetic construct
MRPRLWLLLAAQLTVLHGNSV

SEQ ID NO: 18 - protein, 15 aa, synthetic construct
GGGGSGGGGSGGGGS

SEQ ID NO: 19 - protein, 241 aa, synthetic construct
EVQLVESGGGLVKPGGSLRLSCAASGFSLSNYVMSWVRQAPGKGLEWVATISSGGRYIYYTDSVKGRFTISRDNAKNSLYLQMNSLRAEDTAVYYCARDGSTLYYFDYWGQGTTVTVSSGGGGSGGGGSGGGGSQTVLTQSPATLSLSPGERATLSCSASSSVDYMYWYQQKPGQAPRLLIYDTSNLATGIPARFSGSGSGTDFTLTISSLEPEDFATYYCQQWSSSPPITFGQGTKVEIK

SEQ ID NO: 20 - protein, 241 aa, synthetic construct
QTVLTQSPATLSLSPGERATLSCSASSSVDYMYWYQQKPGQAPRLLIYDTSNLATGIPARFSGSGSGTDFTLTISSLEPEDFATYYCQQWSSSPPITFGQGTKVEIKGGGGSGGGGSGGGGSEVQLVESGGGLVKPGGSLRLSCAASGFSLSNYVMSWVRQAPGKGLEWVATISSGGRYIYYTDSVKGRFTISRDNAKNSLYLQMNSLRAEDTAVYYCARDGSTLYYFDYWGQGTTVTVSS

SEQ ID NO: 21 - protein, 22 aa, synthetic construct
IWAPLAGTCGVLLLSLVITLYC

SEQ ID NO: 22 - protein, 47 aa, synthetic construct
TTTPAPRPPTPAPTIASQPLSLRPEACRPAAGGAVHTRGLDFACDIY

SEQ ID NO: 23 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 467 aa, synthetic construct
QTVLTQSPATLSLSPGERATLSCSASSSVDYMYWYQQKPGQAPRLLIYDTSNLATGIPARFSGSGSGTDFTLTISSLEPEDFATYYCQQWSSSPPITFGQGTKVEIKGGGGSGGGGSGGGGSEVQLVESGGGLVKPGGSLRLSCAASGFSLSNYVMSWVRQAPGKGLEWVATISSGGRYIYYTDSVKGRFTISRDNAKNSLYLQMNSLRAEDTAVYYCARDGSTLYYFDYWGQGTTVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 467 aa, synthetic construct
EVQLVESGGGLVKPGGSLRLSCAASGFSLSNYVMSWVRQAPGKGLEWVATISSGGRYIYYTDSVKGRFTISRDNAKNSLYLQMNSLRAEDTAVYYCARDGSTLYYFDYWGQGTTVTVSSGGGGSGGGGSGGGGSQTVLTQSPATLSLSPGERATLSCSASSSVDYMYWYQQKPGQAPRLLIYDTSNLATGIPARFSGSGSGTDFTLTISSLEPEDFATYYCQQWSSSPPITFGQGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 22 aa, synthetic construct
MLLLVTSLLLCELPHPAFLLIP

SEQ ID NO: 28 - protein, 45 aa, synthetic construct
TTTPAPRPPTPAPTIASQPLSLRPEACRPAAGGAVHTRGLDFACD

SEQ ID NO: 29 - protein, 21 aa, synthetic construct
PFFFCCFIAVAMGIRFIIMVT

SEQ ID NO: 30 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 31 - protein, 24 aa, synthetic construct
IYIWAPLAGTCGVLLLSLVITLYC

SEQ ID NO: 32 - protein, 39 aa, synthetic construct
IEVMYPPPYLDNEKSNGTIIHVKGKHLCPSPLFPGPSKP

SEQ ID NO: 33 - protein, 27 aa, synthetic construct
FWVLVVVGGVLACYSLLVTVAFIIFWV

SEQ ID NO: 34 - protein, 41 aa, synthetic construct
RSKRSRLLHSDYMNMTPRRPGPTRKHYQPYAPPRDFAAYRS